(19) United States
(12) Patent Application Publication
(10) Pub. No.: US 2021/0395358 A1
(43) Pub. Date: Dec. 23, 2021

(54) USE OF CLAZAKIZUMAB TO DESENSITIZE AND IMPROVE RENAL TRANSPLANTATION IN HLA-SENSITIZED PATIENTS

(71) Applicant: CEDARS-SINAI MEDICAL CENTER, Los Angeles, CA (US)

(72) Inventors: Stanley C. JORDAN, Manhattan Beach, CA (US); Ashley VO, Northridge, CA (US); Noriko AMMERMAN, Los Angeles, CA (US); Jua CHOI, San Ramon, CA (US)

(73) Assignee: Cedars-Sinai Medical Center, Los Angeles, CA (US)

(21) Appl. No.: 17/292,025

(22) PCT Filed: Nov. 8, 2019
(86) PCT No.: PCT/US2019/060622

§ 371 Date: May 7, 2021

Related U.S. Application Data
(60) Provisional application No. 62/855,988, filed on Jun. 1, 2019.
(60) Provisional application No. 62/757,676, filed on Nov. 8, 2018.

(51) Int. Cl. C07K 16/24; A61K 35/16; A61K 39/395; A61K 31/522; A61K 31/4196; A61K 31/506; A61K 31/635; A61K 31/436; A61K 31/5377; A61K 31/573; A61P 37/06

(52) U.S. Cl. CPC C07K 16/248; A61K 35/16; A61K 39/3955; A61K 31/522; A61K 31/4196; A61P 37/06; A61K 31/635; A61K 31/436; A61K 31/5377; A61K 31/573; A61K 31/506

(57) ABSTRACT

Methods for desensitization of patients in need of organ transplant are provided Human leukocyte antigen-sensitized patients awaiting incompatible kidney transplant have been treated with clazakizumab to show reduced or eliminated levels of donor-specific antibodies and an improved transplant rate Clazakizumab and variants are provided for use in various embodiments of the methods. In some embodiments, clazakizumab, or its variants, is administered simultaneously or sequentially with intravenous immunoglobulin.

### CROSS-REFERENCE TO RELATED APPLICATIONS

This application includes a claim of priority under 35 U.S.C. § 119(e) to U.S. provisional patent application No. 62/757,676, filed Nov. 8, 2018, and to U.S. provisional patent application No. 62/855,988, filed Jun. 1, 2019, the entireties of which are hereby incorporated by reference.

### FIELD OF INVENTION

This invention relates to therapies and treatment methods for desensitization and improving organ transplantation in sensitized patients.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Causes for the accelerated decline of renal allografts are multifactorial. Recent data indicates a reversal of the long held opinion that calcineurin inhibitor (CNI) toxicity was primarily responsible for the majority of chronic allograft failures. Now, it is recognized that alloimmune responses are responsible for the majority of renal allograft failures which total 5,000 per year in the U.S. The cost associated with failed allografts represents a considerable financial burden to the health care system while decreasing the length and quality of life of those affected. Patients returning to the transplant list after allograft failure now represent the fourth largest category for new patient listings in the U.S. These patients represent a major problem for transplant centers as they are highly-human leukocyte antigen (HLA) sensitized and unlikely to receive another transplant without significant desensitization. There are currently no FDA approved drugs in this category. Development of new therapies to decrease allo-sensitization and improve transplant rates is very important. Today, this represents one of the most important goals of transplant medicine.

HLA molecules are polymorphic. Each HLA molecule expresses polymorphic private epitope(s) and a number of public determinants that represent epitopes shared by more than one HLA molecule. Immunization after previous transplantation, blood transfusion, or pregnancy can lead to the development of HLA specific antibodies. An important responsibility of the immunogenetics laboratory is to identify and analyze HLA specific antibodies that are present in a patient's serum pre- or post-transplantation. The knowledge of the specificity of alloantibodies can help predict the likelihood of finding a crossmatch compatible donor, to avoid transplantation with a donor carrying HLA antigens to which the patient is sensitized to, to select an optimal crossmatch method, and/or to avoid a false positive crossmatch with a donor by excluding clinically irrelevant antibodies.

Antibodies to HLA antigens have a strong impact on mediation of allograft injury and loss and remain a persistent and often impenetrable barrier to successful transplantation for thousands of patients on renal transplant lists worldwide. Pre-formed or de novo donor specific antibodies (DSAs) activate complement, induce endothelial cell proliferation and mediate antibody dependent cytotoxicity (ADCC), which leaves the recipient highly HLA sensitized, suffering from persistent immune attack on the allograft, and results in a progression of interstitial fibrosis, tubular atrophy (IF/TA), and allograft dysfunction and loss. Patients returning to dialysis have little hope of receiving a subsequent transplant and often face a higher risk of death on dialysis. DSAs are also known to accelerate atherosclerosis in the allograft thus hastening the vascular demise of the kidney.

To increase renal transplant rates in sensitized patients, new protocols for HLA desensitization have emerged. These approaches require the application of intravenous immunoglobulin (IVIG), rituximab and plasma exchange (plasmapheresis, PLEX). There is a growing interest in developing new immune-modulatory drugs that are less expensive and more convenient for improving antibody reduction in transplantation.

Existing IVIG-related therapies mainly have two desensitization regimens, i.e., low-dose intravenous immunoglobulin with plasma exchange (IVIG/PLEX) and high-dose IVIG (HD-IVIG). IVIG/PLEX has been used successfully in ABO-incompatible and positive crossmatch (+CMX) living donor renal transplantation, while HD-IVIG has been used to desensitize both living-donor +CMX and highly HLA-sensitized-deceased donor (HS-DD) recipients on the waitlist. HD-IVIG (2 g/kg) in multiple dosing regimens is considered a reasonable approach for desensitization. The B-cell depleting agent, rituximab, is often used in combination with HD-IVIG and IVIG/PLEX protocols. Rituximab in the IVIG/rituximab protocol is shown to be able to modify allo-reactive B-cells and prevent DSA rebound.

A major issue with existing desensitization regimens is in the interpretation of CMX and DSA results when IVIG and rituximab are present in test sera. IVIG given at the dose of 2 gm/kg (maximum 140 grams) will likely interfere with the LUMINEX test for DSA, giving false positive results. This in theory can be avoided by waiting at least 1 month after IVIG administration to perform LUMINEX single antigen bead (LSA) testing since the half-life of IVIG is 30-40 days. Rituximab does not interfere with the LSA testing platforms, but does give “false positive” CDC+ and flow cytometry crossmatch (FCMX)-positive B-cell crossmatches that may be mistakenly interpreted as being due to DSAs. Pronase treatment of B-cells prior to FCMX and CDC testing generally reduces the effect of rituximab, but this is not always dependable.

Alloantibodies are a major deterrent to access to and success of life-saving organ transplants. Despite advancements in desensitization, designing efficient and effective means for removing pathogenic anti-HLA antibodies remains a significant medical challenge. There are a number of notable deficiencies of the existing desensitization protocols. For example, the failure of current therapies to substantially completely remove DSAs before transplantation results in the risk for acute rejection. There is also a risk of rebound DSA formation post-transplant with attendant injury to the allograft, both acute and chronic. And some of current protocols, especially those utilizing complement inhibitors to prevent chronic antibody mediated rejection (cABMR), still fail to deliver desirable outcomes. As such, an unmet medical need exists to improve the ability to reduce or eliminate pre-existing HLA antibodies to a level that would allow patients to receive life-saving organ transplants.

Therefore, it is an objective of the present invention to provide a composition for use in combination with, improving, or in replacement of existing standard treatment of desensitization, so as to improve the solid organ transplant rate for HLA-sensitized subjects.

### SUMMARY OF THE INVENTION

The following embodiments and aspects thereof are described and illustrated in conjunction with compositions and methods which are meant to be exemplary and illustrative, not limiting in scope.

Methods for reducing donor-specific antibody and/or desensitization in a human leukocyte antigen (HLA)-sensitized human subject are provided. The method includes administering to the subject an effective amount of clazakizumab; an IL-6 binding fragment of clazakizumab; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7; where the subject is in need of or has undergone a solid organ transplantation. Various embodiments provide the subject is a human.

In one embodiment, clazakizumab, the IL-6 binding fragment of clazakizumab, or the polypeptide disclosed herein is administered before transplantation. In another embodiment, clazakizumab, the IL-6 binding fragment of clazakizumab, or the polypeptide is administered after transplantation. Yet another embodiment provides clazakizumab, the IL-6 binding fragment of clazakizumab, or the polypeptide is administered both before and after transplantation.

Some embodiments of the disclosed method provide administering a standard-of-care treatment including intravenous immunoglobulin (IVIG) administration, rituximab administration, plasmapheresis, or a combination thereof, in addition to the administration of clazakizumab, the IL-6 binding fragment of clazakizumab, or the polypeptide disclosed herein. In one aspect, the standard-of-care treatment is administered before clazakizumab, the IL-6 binding fragment of clazakizumab, or the polypeptide. In another aspect, the standard-of-care treatment is administered concurrent or after the administration of clazakizumab, the IL-6 binding fragment of clazakizumab, or the polypeptide.

One embodiment provides the method is for desensitizing HLA-sensitized human patients awaiting kidney transplant, where the method includes administering an effective amount of clazakizumab, the IL-6 binding fragment of clazakizumab, or the polypeptide disclosed herein. Another embodiment provides the method for desensitizing HLA-sensitized human patients awaiting kidney transplant includes administering an effective amount of (1) clazakizumab, the IL-6 binding fragment of clazakizumab, or the polypeptide disclosed herein, (2) a standard-of-care treatment, such as IVIG, plasmapheresis, rituximab, or a combination thereof, and optionally (3) an anti-infectious agent.

Other embodiments provide that one or more of the methods is for desensitizing HLA-sensitized human patient for other solid organ transplantation including heart, liver, lung, pancreas, or intestine.

In one embodiment, clazakizumab, the IL-6 binding fragment of clazakizumab, or the polypeptide is administered subcutaneously at an average dose of about 1-5, 5-10, 10-20 or 20-30 mg/time for 1, 2, 3, 4, 5 or 6 times prior to transplantation and 4, 5, 6, 7, 8, 9, 10, 11 or 12 times after transplantation, where the subject has reduced amounts of donor-specific antibodies after the treatment compared to before the treatment. Various aspects provide the post-transplantation clazakizumab, an antigen-binding fragment thereof, or a polypeptide disclosed herein is administered at about a monthly interval. One embodiment provides administering to the subject one dose of clazakizumab, IVIG and plasmapheresis before transplantation, followed by six doses or 12 doses of clazakizumab post-transplantation. Various embodiments provide the disclosed methods include administering clazakizumab, an antigen-binding fragment thereof, or a polypeptide disclosed herein to a human subject that is HLA-sensitized and is in need of or has undergone kidney transplantation, wherein the creatinine level of the subject is lowered after the treatment compared to before the treatment, absence of or no detectable presence of donor-specific antibodies, and/or the subject has no detectable symptoms or evidence of developing antibody-mediated rejection (e.g., no deterioration of allograft function measured by serum creatinine and estimated glomerular filtration rate; no detectable evidence of capillaritis, inflammation or complement (C4d) deposition). A further aspect of the embodiment provides the creatinine level of the subject is lowered and maintained at a lowered level for 1, 2, 3, 4, 5 months or longer concurrent with or following the administration of clazakizumab, an antigen-binding fragment thereof, or a polypeptide disclosed herein.

Pharmaceutical compositions for use in the administration to HLA-sensitized subjects in order to desensitize the subjects and increase transplant rate are also provided. The pharmaceutical compositions contain clazakizumab, the IL-6 binding fragment of clazakizumab, or the polypeptide disclosed herein, as well as pharmaceutically acceptable excipients such as amino acids, sorbitol, and diluents.

Various embodiments provide the use of clazakizumab in patients who are HLA-sensitized and who are awaiting incompatible kidney transplantation, where DSAs, complement dependent cytotoxicity (CDC) and/or antibody dependent cytotoxicity (ADCC) are reduced or eliminated (e.g., DSA reduced or eliminated from the sera). In some embodiments, patients with clazakizumab treatment are appropriate for transplant with less likelihood for antibody reactions.

Some aspects provide one or more of the disclosed methods further includes selecting a mammalian (e.g., human) patient that is HLA sensitized and awaiting incompatible deceased donor (DD) or living donor (LD) renal transplants.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts the DSA profile in the study for subject “ClazaDES01,” who is a 50-year old African American female with a history of end-stage renal disease (ESRD) secondary to biopsy proven focal segmental glomerulosclerosis (FSGS) and who had been on dialysis since November 2008 (i.e., approximately 10 years' of wait-time for B+ blood type) with calculated panel reactive antibodies (cPRA) of 58%. Patient's sensitizing events included pregnancies ×4 and blood transfusion.

FIG. 2 depicts the DSA profile for subject “ClazaDES05” pre- and post-transplantation. (Median fluorescence intensity, MFI). Subject “ClazaDES05” is 36-year old female with a history of ESRD secondary to IgA nephropathy, and she had been on dialysis since June 2008 (i.e., approximately 10 years' of wait-time for A+ blood type), with cPRA 100%. Patient's sensitizing event included previous transplant and blood transfusion. Subject “ClazaDES05” received a deceased donor kidney transplant after 4 doses of clazakizumab. Patient had 2 DSAs pre- and post-transplant (Class I and II). DSA strengths for class I were reduced from MFI>12,500 MFI at transplantation to MFI=0 at 10 days post-transplantation, and for class II from MFI>17,500 at transplantation to MFI>3250 at 10 days post-transplantation. Patient continued with monthly clazakizumab for 6 months post-transplantation as per study protocol.

FIG. 3A depicts the overall C-reactive protein amount in the clazakizumab desensitization study. Overall, C-reactive protein (CRP) was reduced from baseline to nearly zero by the second month. Number of subjects included in the analysis for each time point is indicated in parentheses.

FIG. 3B depicts the individual C-reactive protein amounts in the clazakizumab desensitization study from baseline to the seventh month.

FIG. 4 depicts the sum of MFI over time from before plasmapheresis (PLEX) (pre-PLEX) to the fifth dose of clazakizumab (N=9). Typically, MFI tends to rebound by approximately 1-3 months after completion of PLEX/IVIG. Here, with monthly clazakizumab injection, the sum of MFI remained reduced over time when compared to pre-PLEX. Three patients were transplanted to date. Patients ClazaDES01 and ClazaDES03 were transplanted after the first dose of clazakizumab. Patient ClazaDES05 received a transplant after the fourth dose of clazakizumab.

FIG. 5 is a schematic depiction of an exemplary method for desensitizing HLA-sensitized patients before renal transplantation. Patients will receive up to 6 doses of Clazakizumab while monitoring anti-HLA antibodies (DSA levels), Treg cell and plasmablasts at select time points during the study. For example, DSA levels are collected on all points, including Day 0, except for day 7. The amounts of C-reactive protein (CRP) and quantitative immunoglobulins (QIGs) are collected on all points, including baseline (−15 days), except for day 0. Additionally, the following are collected for baseline (−15 days) and on day 180: CD4+/CD25+/Fox P3+/CD127 low cell numbers (Tregs); Th17+ cell numbers; and CD19+/CD38+/CD27+/IL-6+ (plasmablast). For subjects who do not get transplanted before day 180, pre-transplant, specialized testing will be performed. For subjects who get transplanted before day 180, pre-transplant, specialized testing will be done on transplant day 0. For maintenance, the standard of regimen includes tacrolimus, mycophenolate mofetil, and steroid.

FIG. 6 is a schematic depiction of an exemplary method for post-transplantation prophylaxis and/or treatment to reduce donor-specific antibodies. In an aspect that is subsequent to the pre-transplantation treatment as exemplified in FIG. 5, IVIG and clazakizumab are administered post-transplantation (transplantation day is denoted Day 0 in FIG. 6). The DSA levels are monitored on days 0, 90 and 180; also on day 270 in those who receive a second round of dosing. The levels of CRP and QIGs are collected on days 0, 30, 60, 90, 120 150 and 180; also on 240 and 300 in those who receive a second round of dosing. On day 180 (about 6-month) post-transplant, the following levels are collected: CD4+/CD25+/Fox P3+/CD127 low cell numbers (Tregs); Th17+ cell numbers; and CD19+/CD38+/CD27+/IL-6+ (plasmablast). Viral PCT tests (for cytomegalovirus, Epstein-Barr virus, polyomavirus, BK virus, JC virus, and parvovirus B19) are performed on days 30, 90, 180; also on days 270, 330 if patient receives second round of dosing. For maintenance, the standard of regimen includes tacrolimus, mycophenolate mofetil, and steroid. The “Added plan” includes if patient shows stabilization or improvement in the a) 6M protocol biopsy Banff 2015 read; b) glomerular filtration rate (GFR); c) DSA, patient is to continue clazakizumab monthly for another 6 doses for days around 180, 210, 240, 270, 300 and 330.

FIG. 7 depicts the timeline of treatment on patient “ClazaDES03” in the study in Example and his creatinine level (mg/dL) from before the treatment to after the treatment.

FIG. 8 shows the renal transplant biopsy (including tubular injury, arteriosclerosis and very focal tubulitis) at about 2 months following transplantation of patient “ClazaDES03.”

FIG. 9 shows the renal transplant biopsy (including acute tubular necrosis, rare isometric vacuoles, and mild tubulointerstitial inflammation) at about 6 months following transplantation of patient “ClazaDES03.”

FIGS. 10A and 10B depict flow panel reactive antibody test (flow-PRA) class I/class II, respectively, at pre-desensitization and at post-transplantation (post-Tx).

FIG. 11 depicts HLA class I & class II antibodies of various markers, and the overall amount, at pre-desensitization and at the last follow-up (F/U) about 6-12 months post-transplantation for subject DES03 receiving clazakizumab. No DSA was detected at-transplantation and post-transplantation.

FIGS. 12A-12C depict HLA class I & class II antibodies of various markers, and the overall amount, at pre-desensitization and post-transplantation for subject DES05 having received clazakizumab.

FIGS. 13A-13C depict HLA class I & class II antibodies of various markers, and the overall amount, at pre-desensitization and post-transplantation for subject DES07 having received clazakizumab.

FIG. 14 depicts HLA class I & class II antibodies of various markers, and the overall amount, at pre-desensitization and post-clazakizumab for subject DES02 (non-transplanted).

FIG. 15 depicts HLA class I & class II antibodies of various markers, and the overall amount, at pre-desensitization and post-clazakizumab for subject DES09 (non-transplanted).

FIG. 16 depicts HLA class I & class II antibodies pre- and post-clazakizumab for all study patients (N=10).

FIGS. 17A-17C depict HLA class I antibodies (FIG. 17A), class II antibodies (FIG. 17B) pre- and post-clazakizumab for transplanted patients (N=8) (combined comparison in FIG. 17C).

FIG. 18 depicts DSA(s) for individual patient for class I & class II: pre-desensitization, at-transplant and post-transplant (N=8).

FIG. 19 depicts mean DSAs MFI for class I & class II: pre-desensitization, at-transplant & post-transplant.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “transplant rate” generally refers to the number of patients who undergo transplant for every 100 patients who are on the waiting list during a year. In some aspects, it is a measure of how frequently patients on a program's waiting list undergo transplant. To make it easier to compare numbers, in some aspects the rate is given “per 100 patient-years,” which means that the rate is normalized to what it would be there were 100 patients on the list for a year. For example, a transplant rate of 5 per 100 patient-years means that for every 100 patients on the list during a year, 5 transplants are performed. Because this is a normalized rate, the number may include a decimal, for example, 5.1 per 100 patient-years. This means a slightly more than 5 patients are expected to undergo transplant for every 100 patients on the list during a year.

A positive crossmatch (+CMX) indicates the presence of donor specific alloantibodies (DSA) in the serum of a potential recipient, and is often associated with a rate of graft loss that exceeds 80%.

“HLA-sensitized (HS) patient” in the Example study refers to patients awaiting kidney transplantation on the United Network for Organ Sharing (UNOS) waitlist whose calculated panel reactive antibodies (cPRA) or percentage of likely cross-match incompatible donors is ≥50%, who in various embodiments also has demonstrable DSA using LUMINEX bead technology and a history of sensitizing events (e.g., previous transplants, blood transfusions and/or pregnancies). The presence of HLA specific antibodies can be determined by testing patient's sera against cells from a panel of HLA typed donors or against solubilized HLA antigens attached to solid supports. Generally, HLA-sensitized patients refer to patients whose cPRA is no less than 10%, 20%, 30%, 40% or 50%.

A “subject” means a human or an animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

The terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as weight loss or muscle loss resulting from cancer cachexia. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

The term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g., papain cleavage) or via chemical cleavage of intact antibodies. An antibody can be a chimeric, humanized or human antibody. An antibody can be an IgG1, IgG2, IgG3 or IgG4 antibody. In some aspects, an antibody herein has an Fc region that has been modified to alter at least one of effector function, half-life, proteolysis, or glycosylation.

The term “antibody fragment,” refers to a protein fragment that comprises only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and CH1 domains; (ii) the Fab′ fragment, which is a Fab fragment having one or more cysteine residues at the C-terminus of the CH1 domain; (iii) the Fd fragment having V_{H} and CH1 domains; (iv) the Fd′ fragment having V_{H} and CH1 domains and one or more cysteine residues at the C-terminus of the CH1 domain; (v) the Fv fragment having the V_{L} and V_{H} domains of a single arm of an antibody; (vi) the dAb fragment which consists of a V_{H} domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv); (x) “diabodie” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-CH1-V_{H}-CH1) which, together with complementary light chain polypeptides, form a pair of antigen binding regions. An antibody or antibody fragment can be scFvs, camelbodies, nanobodies, IgNAR (single-chain antibodies derived from sharks) and Fab, Fab′ or F(ab′)₂ fragment.

“Selectively binds” or “specifically binds” refers to the ability of an antibody or antibody fragment thereof described herein to bind to a target, such as a molecule present on the cell-surface, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

“Ineffective” treatment refers to when a subject is administered a treatment and there is less than 5%, improvement in symptoms. If specifically provided for in the claim, ineffective treatment can refer to less than 1%, 2%, 3%, 4%, 6%, 7%, 8%, 9% or 10% improvement in symptoms.

“Adverse Events,” an adverse event is any unfavorable and unintended sign, symptom, or disease temporally associated with the use of an investigational medicinal product (IMP) or other protocol-imposed intervention, regardless of attribution. An adverse event can be any unfavorable and unintended sign (including an abnormal laboratory finding), symptom, or disease temporally associated with the use of a medicinal product, whether or not considered related to the medicinal product. Surgical procedures are not adverse events; they are therapeutic measures for conditions that require surgery. However, the condition for which the surgery is required is an adverse event, if it occurs or is detected during the Study in the Example. Planned surgical measures and the condition(s) leading to these measures are not adverse events, if the condition(s) was (were) known before the start of Study treatment. In the latter case, the condition should be reported as medical history.

A preexisting condition is one that is present at the start of the Study. Preexisting conditions that worsen during the study are considered adverse events. A preexisting condition should be recorded as an adverse event if the frequency, intensity, or the character of the condition worsens during the Study period.

“Abnormal Test Findings,” an abnormal test finding that meets any one of the criteria below should be considered an adverse event:

Test result is associated with accompanying symptoms;

Test result requires additional diagnostic testing or medical/surgical intervention;

Test result leads to a change in Study treatment dosing (e.g., dose modification, interruption, or permanent discontinuation) or concomitant drug treatment (e.g., addition, interruption, or discontinuation) or any other change in a concomitant medication or therapy;

Test result leads to any of the outcomes included in the definition of a serious adverse event (note: this would be reported as a serious adverse event);

Test result is considered an adverse event by the Investigator.

Laboratory results that fall outside the reference range and do not meet one of the criteria above should not be reported as adverse events. Repeating an abnormal test, in the absence of the above conditions, does not constitute as adverse event. Any abnormal test result that is determined to be an error does not require reporting as an adverse event.

“Serious Adverse Event,” a serious adverse event (SAE) is any untoward medical occurrence that at any dose:

Is fatal (results in death);

Is life-threatening: The patient was at immediate risk of death from the adverse event as it occurred. This does not include an event that, had it occurred in a more severe form or was allowed to continue, might have caused death;

Requires inpatient hospitalization or prolongation of existing hospitalization;

Results in persistent or significant disability/incapacity;

Is a congenital anomaly/birth defect (in the child of a patient who was exposed to the Study treatment);

An important medical event that may not result in death, be life threatening, or require hospitalization may be considered an SAE when, based upon appropriate medical judgment, the event may jeopardize the subject and may require medical or surgical intervention to prevent one of the outcomes listed in this definition. Examples of such events are intensive treatments in an emergency room or at home for allergic bronchospasm, blood dyscrasias, or convulsions that do not result in hospitalization, or development of drug dependency or drug abuse.

Adverse events resulting in hospitalization are considered serious. Any adverse event resulting in initial admission to a healthcare facility or transfer within the hospital to an acute/intensive care unit is considered serious.

Hospitalization or prolongation of hospitalization in the absence of a precipitating, clinical adverse event is not in itself a serious adverse event. Examples that are not considered a serious adverse event include: (1) Admission for treatment for a preexisting condition not associated with the development of a new adverse event or with a worsening of the preexisting condition, (2) social or administrative admission, (3) optional admission not associated with a precipitating clinical adverse event, (4) pre-planned treatments or surgical procedures should be noted in baseline documentation.

The Investigator's assessment of severity is required for all adverse events. The following criteria are used to assess severity:

Mild: discomfort noticed but no disruption of normal daily activity;

Moderate: discomfort sufficient to reduce or affect daily activity; and

Severe: inability to work or perform daily activity.

To clarify the difference between the terms “serious” and “severe,” which are not synonymous, note that the term “severe” is often used to describe the intensity (severity) of a specific event, such as mild, moderate, or severe myocardial infarction. The event itself, however, may be of relatively minor medical significance, such as a severe headache. This is not the same as “serious” which is based on patient/event outcome or action criteria usually associated with events that pose a threat to a patient's life or functioning. Seriousness (not severity) serves as a guide for defining regulatory reporting obligations.

Causality assessment is the determination of whether there exists a reasonable possibility that the Study treatment caused or contributed to an adverse event. “Not related” describes that temporal relationship to Study treatment administration is missing or implausible, or there is evidence of another cause. “Unlikely related” describes that temporal relationship to Study treatment administration makes a causal relationship improbable; and other drugs, chemicals, or underlying disease provide plausible explanations. “Possibly related” describes that reasonable time sequence to administration of Study treatment, but the event could also be explained by concurrent disease of other drugs or chemicals. Information on drug withdrawal may be lacking or unclear. “Definitely related” describes plausible time relationship to Study treatment administration; event cannot be explained by concurrent disease or other drugs or chemicals. The response to withdrawal of the drug (dechallenge) should be clinically plausible. The event must be definitive pharmacologically or phenomenologically, using a satisfactory re-challenge procedure if necessary.

Interleukin-6 is an important mediator of inflammation and the development, maturation, and activation of T-cells, B-cells and plasma cells. Excessive IL-6 production has been linked to a number of human diseases characterized by excessive and unregulated antibody production and autoimmunity.

The disclosed method for desensitizing an HLA-sensitized subject, reducing the amount of donor specific antibodies, and/or improving organ transplant rate or transplant survival includes administering to the subject an effective amount of clazakizumab, or an antibody or antigen-binding fragment thereof which shares at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence homology (identical) to clazakizumab or the complementarity-determining regions (CDRs) of clazakizumab. Some embodiments provide one or more of the methods further includes administering an effective amount of IVIG or plasmapheresis.

Clazakizumab is a glycosylated humanized (from a rabbit parental antibody) monoclonal antibody targeting interleukin-6. The peptide sequence and structural information of clazakizumab are available from IMGT/mAb-db record #414. BLAST peptide sequence analysis reveals identical matches with peptides claimed in U.S. Pat. No. 8,062,864, which is herein incorporated by reference in its entirety. Further description of clazakizumab and its variants is shown in U.S. Pat. No. 7,935,340, which is herein incorporated by reference in its entirety, whose antibodies or antibody fragments are suitable for the methods disclosed herein of reducing or eliminating donor specific antibodies in a subject in need of or having undergone allograft transplantation. For example, the antibody has V_{H} polypeptide containing CDR1, CDR2, and CDR3 polypeptides which respectively are contained in SEQ ID NO: 1 (for V_{H} CDR1), SEQ ID NO: 2 or 3 (for V_{H} CDR2), and SEQ ID NO: 4 (for V_{H} CDR3), and has V_{L} polypeptide containing CDR1, CDR2, and CDR3 polypeptides which respectively are contained in SEQ ID NO: 5, 6, and 7. The anti-human IL-6 antibody includes a variable heavy chain contained in SEQ ID NO: 8, 9 or 10, and a variable light chain contained in SEQ ID NO: 11 or 12.

(SEQ ID NO: 1)
Asn-Tyr-Tyr-Val-Thr
(SEQ ID NO: 2)
Ile-Ile-Tyr-Gly-Ser-Asp-Glu-Thr-Ala-
Tyr-Ala-Thr-Trp-Ala-Ile-Gly
(SEQ ID NO: 3)
Ile-Ile-Tyr-Gly-Ser-Asp-GIu-Thr-Ala-
Tyr-Ala-Thr-Ser-Ala-Ile-Gly
(SEQ ID NO: 4)
Asp-Asp-Ser-Ser-Asp-Trp-Asp-Ala-Lys-
Phe-Asn-Leu
(SEQ ID NO: 5)
Gln-Ala-Ser-Gln-Ser-Ile-Asn-Asn-Glu-
Leu-Ser
(SEQ ID NO: 6)
Arg-Ala-Ser-Thr-Leu-Ala-Ser
(SEQ ID NO: 7)
Gln-Gln-Gly-Tyr-Ser-Leu-Arg-Asn-Ile-
Asp-Asn-Ala.
A variable heavy chain sequence is
set forth in SEQ ID NO: 8
METGLRWLLLVAVLKGVQCQSLEESGGRLVTPGTPL
TLTCTASGFSLSNYYVTWVRQAPGKGLEWIGIIYGS
DETAYATWAIGRFTISKTSTTVDLKMTSLTAADTAT
YFCARDDSSDWDAKFNLWGQGTLVTVSSASTKGPSV
FPLAPSSKSTSGGTAALGCLVK.
A substituted variable heavy
chain sequence is set forth
in SEQ ID NO: 9
EVQLVESGGGLVQPGGSLRLSCAASGFSLSNYYVTW
VRQAPGKGLEWVGIIYGSDETAYATWAIGRFTISRD
NSKNTLYLQMNSLRAEDTAVYYCARDDSSDWDAKFN
L.
Another substituted variable heavy
chain sequence is set forth
in SEQ ID NO: 10
EVQLVESGGGLVQPGGSLRLSCAASGFSLSNYYVTW
VRQAPGKGLEWVGIIYGSDETAYATSAIGRFTISRD
NSKNTLYLQMNSLRAEDTAVYYCARDDSSDWDAKFN
L.

Clazakizumab is a genetically engineered humanized immunoglobulin G1 (IgG1) antibody that binds to human IL-6 with an affinity of about 4 pM. Using multiple assays for signaling and cellular functions in response to IL-6 alone (to measure classical signaling) and a combination of IL-6 and sIL-6R (to measure trans-signaling), it was demonstrated that clazakizumab is a potent and full antagonist of IL-6-induced signaling as measured by phosphorylation of signal transducer and activator of transcription 3 (STAT3), as well as cellular functions such as cell proliferation, differentiation, activation, B-cell production of immunoglobulins, and hepatocyte production of acute phase proteins (C-reactive protein [CRP] and fibrinogen). In addition, clazakizumab is shown to be a competitive antagonist of IL-6-induced cell proliferation.

Clazakizumab exhibits a broad range of immunomodulatory actions that can address destructive allo-antibody response to allografts and be useful as a desensitization agent to improve rates of renal transplantation for highly-HLA sensitized patients. Clazakizumab has been evaluated extensively in patients with rheumatoid arthritis, but has not yet been approved by the FDA for any condition. Since the introduction of IL-6/IL-6R blocking drugs, reports indicate that inhibition of the IL-6/IL-6R pathway may have significant benefits in systemic lupus erythematosus (SLE) and other vasculitic disorders and reduces antibody producing cells in treated patients. There is currently no information of clazakizumab in highly sensitized patients awaiting incompatible (HLAi) transplants or for treatment of antibody-mediated rejection.

To date, no studies with clazakizumab have been conducted in subjects with highly sensitized patients undergoing renal transplant, despite clazakizumab studies in healthy subjects and in subjects with rheumatoid arthritis (RA), psoriatic arthritis (PsA), Crohn's disease, graft-versus-host disease (GVHD), and oncology.

Various embodiments provide one or more methods for reducing donor-specific antibodies in an HLA-sensitized subject, characterized by having a calculated panel reactive antibodies (cPRA) or percentage of likely cross-match incompatible donors of at least 10%, 20%, 30%, 40%, 50%, 60%, or 70%, where the methods include administering an effective amount of clazakizumab; antigen-binding fragment thereof; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, to the subject before renal transplantation, after renal transplantation, or both before and after renal transplantation. Various embodiments of the methods provide administering an effective amount of an anti-human IL-6 antibody or antibody fragment which includes a variable heavy chain in SEQ ID NO: 8, 9 or 10 and a variable light chain in SEQ ID NO: 11 or 12 to a subject that has had HLA-sensitization before or after the subject receives an allograft transplant, so as to reduce or eliminate donor specific antibodies in the subject after the allograft transplantation. Some embodiments of the methods further include selecting a subject that has a calculated panel reactive antibodies (cPRA) or percentage of likely cross-match incompatible donors of at least 10%, 20%, 30%, 40%, 50%, 60%, or 70%. Some embodiments of the methods further include performing a kidney transplant in the subject. Further embodiments of the methods are featured by a reduction of donor-specific antibodies after the kidney transplantation, due to the administration of clazakizumab or antigen-binding fragment thereof; or featured by no detectable amount of donor-specific antibodies starting from about one month, two months, three months, four months, five months, or six months after the kidney transplantation, due to the administration of clazakizumab or antigen-binding fragment thereof.

Various embodiments provide one or more methods for reducing donor-specific antibodies in an HLA-sensitized subject, where the methods include administering an effective amount of (1) IVIG and (2) an effective amount of clazakizumab; an IL-6 binding fragment of clazakizumab; or a polypeptide having V_{H} polypeptide including polypeptides of CDR1 contained in SEQ ID NO:1, CDR2 contained in SEQ ID NO: 2 or 3, and CDR3 contained in SEQ ID NO:4, and having V_{L} polypeptide including polypeptides of CDR1 contained in SEQ ID NO: 5, CDR2 contained in SEQ ID NO: 6, and CDR3 contained in SEQ ID NO:7. In one embodiment, IVIG is administered prior to or concurrent with clazakizumab; an IL-6 binding fragment of clazakizumab; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. In some aspects, the HLA-sensitized subject is to receive a solid organ transplant—in one aspect the solid organ is from a crossmatch donor, i.e., the HLA-sensitized subject contains pre-transplantation antibodies that are against the HLA from the organ of the donor; and in another aspect, the solid organ is not from a positive crossmatch donor. In other aspects, the one or more methods further include performing a solid organ transplant, in addition to the administration of an effective amount of (1) IVIG and (2) an effective amount of clazakizumab; an IL-6 binding fragment of clazakizumab; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7.

Various embodiments of the methods for reducing donor-specific antibodies in an HLA-sensitized subject include administering an effective amount of the combination of (1) IVIG and clazakizumab; (2) an effective amount of the combination of IVIG and an IL-6-binding fragment of clazakizumab; or (3) an effective amount of the combination of IVIG and a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. In one embodiment, IVIG is administered prior to or concurrent with clazakizumab; an IL-6 binding fragment of clazakizumab; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. In another embodiment, IVIG is further administered immediately before, during or after a solid organ transplantation in the subject.

Yet more embodiments provide a method for reducing donor-specific antibodies in an HLA-sensitized subject, where the method includes administering (1) an effective amount of the combination of IVIG, plasmapheresis and clazakizumab; (2) an effective amount of the combination of IVIG, plasmapheresis and an IL-6-binding fragment of clazakizumab; or (3) an effective amount of the combination of IVIG, plasmapheresis and a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. In one embodiment, IVIG and plasmapheresis are administered prior to or concurrent with clazakizumab; an IL-6 binding fragment of clazakizumab; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7.

In various embodiments, desensitization due to clazakizumab results in (1) transplantation in 8 of 10 patients in the Study in Example; (2) significant reduction of HLA specificities although cPRA did not significantly change; and (3) reduction of DSA at transplant and post-transplant with continued administration of an effective amount of clazakizumab.

In further embodiments, an anti-IL-6 treatment (e.g., clazakizumab administration) has a significant impact on reducing MFI levels of class II/class II HLA antibodies; thereby increasing transplant rates for HLA-sensitized patients or transplant survival likelihood or percentage for an individual HLA-sensitive patient. Anti-IL-6 mediates this through reduction of IL-6 producing plasma cell (anti-HLA). Post-treatment, anti-IL-6 therapy lowers or eliminates DSA levels and prevent de novo DSA generation. Further aspects provide that no patient receiving clazakizumab and a solid organ transplant has developed antibody-mediated rejection of the transplant.

Various embodiments provide that the disclosed methods include identifying HLA-sensitized patients in need of solid organ transplant, administering PLEX and IVIG and monthly doses of clazakizumab, performing a solid organ transplantation (e.g., kidney transplantation), administering an induction therapy such as alemtuzumab and/or THYMOGLOBULIN (an anti-thymocyte globulin), and administering immunosuppression therapy such as tacrolimus, CELLCEPT (mycophenolate mofetil), and tapering prednisone. In one aspect, the rate of transplantation for HLA-sensitized subjects after desensitization with clazakizumab and PLEX/IVIG is at least 50%, 55%, 60%, 65%, 70%, 75%, 80%, 85%, 90%, or 95%. In another aspect, the time to transplantation after completion of pre-transplant desensitization with clazakizumab and PLEX/IVIG for an HLA-sensitized subject is from 0 day to one week, one week to one month, one month to three months, three months to six months, six months to one year, one year to two years, or longer. In a further aspect, previously HLA-sensitized subjects having received desensitization treatment with an effective amount of clazakizumab, optionally in combination with PLEX/IVIG, and having received an allograft kidney transplant, are at least 95%, 90%, 85%, 80%, 75%, or 70% free from signs or symptoms of antibody-mediated rejection of the transplant. Further aspects of the methods include that the subject does not have rheumatoid arthritis (RA), psoriatic arthritis (PsA), Crohn's disease, graft-versus-host disease (GVHD), a cancer, or a combination thereof. Additional aspects of the methods further include selecting a subject that does not have or has not had rheumatoid arthritis (RA), psoriatic arthritis (PsA), Crohn's disease, graft-versus-host disease (GVHD) or a cancer and that is HLA-sensitized and in need of or having undergone a solid organ (e.g., kidney) transplantation, for the methods of reducing and/or eliminating donor specific antibodies.

Various embodiments provide one or more of the disclosed methods further include performing one or more assays for the presence or absence of infections related to cytomegalovirus, Epstein-Barr virus, polyomavirus, BK virus, JC virus, parvovirus B19, or a combination thereof with the subject before and/or after allograft transplantation. In other embodiments, one or more of the disclosed methods are featured that the subject has no detectable amount of infection related to cytomegalovirus, Epstein-Barr virus, polyomavirus, BK virus, JC virus, parvovirus B19, or a combination thereof, before and/or after the allograft transplantation. Further embodiments provide the subject in one or more of the disclosed post-transplantation clazakizumab methods does not have chronic antibody-mediated rejection of the solid organ transplant or has been tested for the absence of evidence of chronic antibody-mediated rejection.

Various embodiments of the methods for reducing or eliminating donor-specific antibodies in, and/or desensitizing, an HLA-sensitized subject in need of or having undergone an allograft transplantation include administering an effective amount of clazakizumab, an IL-6 binding fragment of clazakizumab, a polypeptide containing a variable heavy chain of SEQ ID NO: 8, 9 or 10 and a variable light chain of SEQ ID NO: 12 or 12, or a polypeptide containing a variable heavy chain with CDR1 of SEQ ID NO:1, CDR2 of SEQ ID NO: 2 or 3, and CDR3 of SEQ ID NO: 4 and a variable light chain with CDR1 of SEQ ID NO:5, CDR2 of SEQ ID NO:6 and CDR3 of SEQ ID NO: 7, in one or more doses over time, wherein (1) the subject has or has had pre-formed donor specific antibodies (DSAs) before the allograft transplantation and/or developed DSAs after the allograft transplantation, (2) the subject has a calculated panel reactive antibodies of 50% or greater, (3) the subject has a high strength of donor-specific antibodies such as determined by single antigen LUMINEX bead assay and expressed as mean fluorescence intensity (MFI) of greater than 9,000, 10,000, 11,000, 12,000 or higher for class I or class II, (4) or the subject has had one or more pregnancies, blood transfusion and/or previous transplantations. In one aspect, the subject has one of the mentioned features. In another aspect, the subject has two of the mentioned features. In yet another aspect, the subject has three of the mentioned features. In yet another aspect, the subject has four of the mentioned features.

Additional embodiments of the methods disclosed herein include one or more steps of immune monitoring before and/or after the allograft transplantation. In various aspects, the methods of reducing or eliminating donor specific antibodies in a subject having had pre-formed DSAs, a cPRA of 50% or greater or a high strength of DSAs before an allograft transplantation, and the subject subsequently having undergone the allograft transplantation (e.g., the allograft is HLA incompatible for the subject), include (i) administering an effective amount of clazakizumab, an IL-6 binding fragment of clazakizumab, or a polypeptide disclosed above, in one or more doses; (ii) conducting (a) immune monitoring of the subject such as assaying the subject's blood samples to quantify Treg, Tfh, Th17, B-cell, IL-6, CRP, plasma cells, plasmablast IgG levels, or a combination thereof, (b) biopsy assessment of the transplant, (c) measuring glomerular filtration rate, and/or (d) measuring amount of DSA in the subject, individually for one or more times, for example, each time following the one or more doses of the clazakizumab, the IL-6 binding fragment of clazakizumab or the polypeptide, over a period of time such as 1 month, 2 months, 3 months, 4 months, 5 months, 6 months, 7 months, 8 months, 9 months, 10 months, 11 months, 12 months, 15 months, 18 months, 24 months or longer; and optionally (iii) when (a) the immune monitoring indicates an improvement in immune reactivity such as characterized by a decreased level of CRP, Treg, Tfh, Th17, B-cell, IL-6, plasma cells, or plasmablast IgG, compared to a previous immune monitoring or to a baseline measurement at or before the allograft transplantation, or a comparable level of CRP, Treg, Tfh, Th17, B-cell, IL-6, plasma cells, or plasmablast IgG level, relative to a healthy subject or a subject having been desensitized, when (b) the biopsy assessment of the transplant indicates absence of cell-mediated and antibody-mediated rejection, absence or reduced evidence of allograft dysfunction (e.g., determined by C4d staining and transplant glomerulopathy, using Banff 2015 criteria), and/or improvement according to Banff 2015 criteria, (c) when glomerular filtration rate is stabilized, e.g., similar or reduced level compared to the last measurement or to prior to the transplantation, or (d) when DSA amount is stabilized, e.g., similar or reduced level compared to the last measurement or to prior to the transplantation, then discontinuing or limiting further administration of the clazakizumab, the IL-6 binding fragment of clazakizumab, or the polypeptide to no more than for another six months; when the immune monitoring indicates that the immune reactivity such as described above has not improved or the amounts of glomerular filtration rate or DSA is not stabilized, then continuing administering the clazakizumab, the IL-6 binding fragment of clazakizumab, or the polypeptide or at an adjusted dosage; and when the biopsy assessment of the transplant indicates presence of cell-mediated and/or antibody-mediated rejection, then administering a standard-of-care treatment to treat the rejection, such as IVIG, plasmapheresis, or both. In some instances, the steps of (ii) and (iii) are repeated for one, two, three, four, five, six, seven, eight, nine or ten times, or continued as needed, or until the improvement, stabilization or even cure is observed.

In some aspects, if evidence of antibody mediated rejection is observed, the subject is directed to treatment for antibody-mediated rejection. In some aspects, after the steps of (i) and (ii), no administration of more doses of the clazakizumab, the IL-6 binding fragment of clazakizumab, or the polypeptide is performed for a period of time (“break”) such as 1 week, 2 weeks, 3 weeks, 4 weeks, 2 months, and 3 months, and after the “break”, immune reactivity is monitored or biopsy of the allograft is assessed, and depending on results such as characterized in step (iii), one skilled in the art will discontinue or continue the administration of the clazakizumab, the IL-6 binding fragment of clazakizumab, or the polypeptide to further reduce or eliminate DSAs in the subject.

Dosage

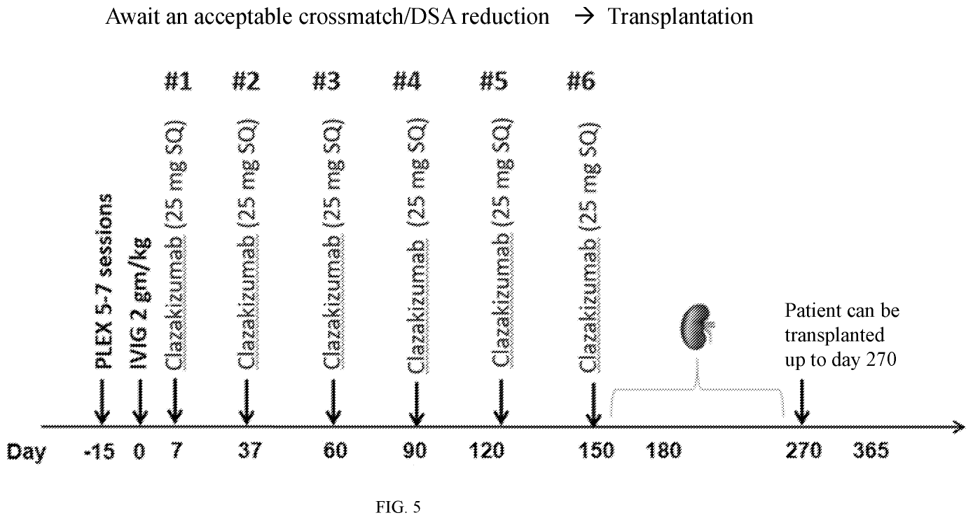

In one embodiment, a method for reducing donor-specific antibodies and HLA desensitization in a subject (e.g., human subject) includes administering an effective amount of clazakizumab or an antibody-binding fragment of clazakizumab prior to transplantation (e.g., at about 25 mg/dose subcutaneously, every 4 weeks for up to six doses). In one embodiment, a method for reducing donor-specific antibodies and HLA desensitization in a subject (e.g., human subject) includes, prior to transplantation, administering plasma exchange (or plasmapheresis) for five, six, or seven sessions followed by an effective amount of IVIG (e.g., at about 2 g/kg of subject, for a maximum of 140 g), and administering an effective amount of clazakizumab (e.g., at about 25 mg subcutaneously, every 4 weeks for up to six doses). In a further embodiment, this method includes transplanting an allograft to the subject. In a further aspect, the transplantation of the allograft is between the last dosing of clazakizumab and about 270 days from the administration of IVIG. This is depicted in FIG. 5. In one aspect, an effective amount of clazakizumab for reducing the level of DSA in a HLA-sensitized subject is about 12.5 mg/dose for 4, 5, 6 or more dose. In one aspect, an effective amount of clazakizumab for reducing the level of DSA in a HLA-sensitized subject is about 25 mg/dose for 4, 5, 6 or more dose. In one aspect, an effective amount of clazakizumab for reducing the level of DSA in a HLA-sensitized subject is not 25 mg/dose at monthly doses of 4, 5 or 6 doses.

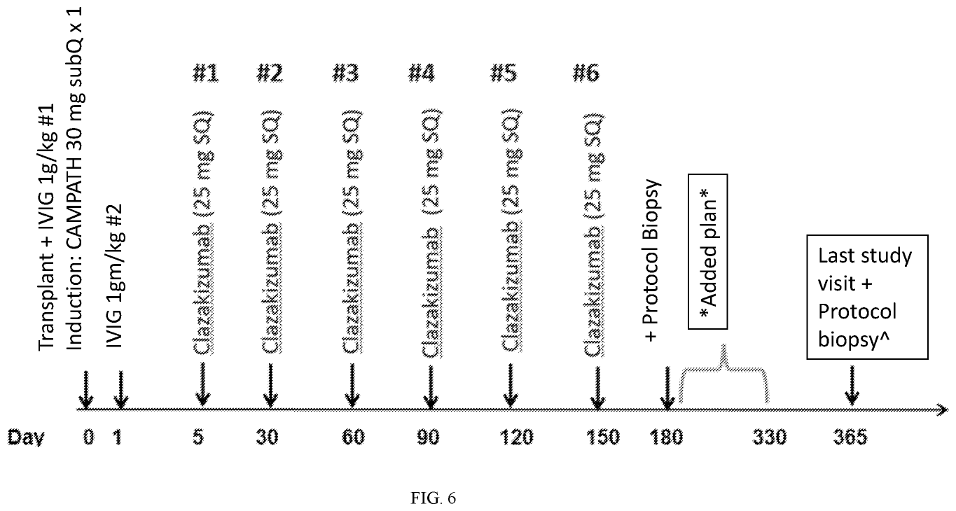

In another embodiment, a method for reducing donor-specific antibodies and HLA desensitization in a subject (e.g., human subject) includes administering an effective amount of clazakizumab or an antigen-binding fragment thereof after transplantation, (e.g., at about 25 mg subcutaneously, every 4 weeks starting at about 5 to 7 days post-transplant, for up to six doses). In a further embodiment, this method includes administering an additional effective amount of clazakizumab (e.g., for another 6 doses, up to day 330 post-transplant). This is depicted in FIG. 6. In one aspect, an effective amount of clazakizumab for reducing the level of DSA after a solid organ transplant in a HLA-sensitized subject is about 12.5 mg/dose for 1, 2, 3, 4, 5 or more doses. In one aspect, an effective amount of clazakizumab for reducing the level of DSA after a solid organ transplant in a HLA-sensitized subject is about 25 mg/dose for 1, 2, 3, 4, 5 or more doses. In one aspect, an effective amount of clazakizumab for reducing the level of DSA after a solid organ transplant in a HLA-sensitized subject is not 25 mg/dose administered every 4 weeks.

In another embodiment, a method for reducing donor-specific antibodies and HLA desensitization in a subject (e.g., human subject) includes administering an effective amount of clazakizumab or an antibody-binding fragment of clazakizumab prior to transplantation (e.g., at about 25 mg/dose subcutaneously, every 4 weeks for up to six doses), and administering an effective amount of clazakizumab or an antigen-binding fragment thereof after transplantation, (e.g., at about 25 mg subcutaneously, every 4 weeks starting at about 5 to 7 days post-transplant, for up to six doses).

Some embodiments of these methods provide assaying the biopsy from the patient, and confirming a stabilized level of glomerular filtration rate (GFR) over time (e.g., less than 10%, 20%, or 30% variations across two, three, or four consecutive biopsies) and a low level (e.g., at less than 10%, 20% or 30%) of DSA compared prior to desensitization treatment, or compared to an earlier biopsy performed after the transplantation. In some embodiments when the level of GFR is not stabilized or the DSA level is high, the method further includes repeated administration of an effective amount of IVIG and clazakizumab, until the level of GFR is stabilized and the level of DSA is low.

Yet another embodiment provides a method for reducing donor-specific antibodies and HLA desensitization in a highly HLA-sensitized subject (e.g., human subject) includes, prior to transplantation, administering plasma exchange (or plasmapheresis); prior to, during, or subsequent to transplantation, administering an effective amount of IVIG; and prior to, subsequent to, or both, administering an effective amount of clazakizumab to the subject, wherein the subject has a stabilized level of glomerular filtration rate (GFR) over time (e.g., less than 10%, 20%, or 30% variations across two, three, or four consecutive biopsies) and a low level (e.g., at less than 10%, 20% or 30%) of DSA compared prior to desensitization treatment.

Following the administration of clazakizumab as a 1-hour IV infusion, the pharmacokinetics of clazakizumab was linear over the dose ranges of 30 mg to 640 mg in healthy subjects and 80 mg to 320 mg in subjects with rheumatoid arthritis (RA) as indicated by consistent clearance at these dose levels. The T-half of clazakizumab at all doses was very similar in healthy male subjects and in subjects with RA and was consistent with that expected for a humanized IgG1 antibody. Across the doses studied, the mean T-half of clazakizumab ranged from 19.5 to 31.0 days in healthy male subjects and from 26.4 to 30.9 days in subjects with RA. The T-half of clazakizumab after SC administration in healthy male subjects was similar to the IV administration. In a Phase 1 study comparing IV and SC dosing in healthy male subjects, the mean T-half of clazakizumab was 30.7 days after a single IV dose and 31.1 to 33.6 days after SC administration. The bioavailability of clazakizumab after SC administration was 60% of the IV formulation. As expected, Cmax was lower and Tmax was longer for the SC administration relative to IV administration.

Population PK analysis of the data from clinical studies in RA, PsA and healthy subjects have indicated that body weight affects the PK of clazakizumab such that both clearance and central volume of distribution increase with increasing body weight. Therefore, heavier subjects will have lower drug exposure compared with less heavy subjects.

The effective amount of clazakizumab for a subject may be investigated or limited based on safety evaluations. Safety evaluations include medical interviews, recording of adverse events, physical examinations, blood pressure, and laboratory measurements. Subjects are generally evaluated for adverse events (all grades), serious adverse events, and adverse events requiring study drug interruption or discontinuation at each study visit for the duration of their participation in the study.

In some embodiments, the effective amounts of clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, can be in the range of about 10-50 μg/dose, 50-100 μg/dose, 100-150 μg/dose, 150-200 μg/dose, 100-200 μg/dose, 200-300 μg/dose, 300-400 μg/dose, 400-500 μg/dose, 500-600 μg/dose, 600-700 μg/dose, 700-800 μg/dose, 800-900 μg/dose, 900-1000 μg/dose, 1000-1100 μg/dose, 1100-1200 μg/dose, 1200-1300 μg/dose, 1300-1400 μg/dose, 1400-1500 μg/dose, 1500-1600 μg/dose, 1600-1700 μg/dose, 1700-1800 μg/dose, 1800-1900 μg/dose, 1900-2000 μg/dose, 2000-2100 μg/dose, 2100-2200 μg/dose, 2200-2300 μg/dose, 2300-2400 μg/dose, 2400-2500 μg/dose, 2500-2600 μg/dose, 2600-2700 μg/dose, 2700-2800 μg/dose, 2800-2900 μg/dose or 2900-3000 μg/dose, for a total of one, two, three, four, five, six, seven, eight, nine, ten, 11, 12, 13, 14, 15 or more doses, or as needed to continue reducing the amount of DSA in the subject, and administered at a frequency of daily, weekly, biweekly, monthly, or bimonthly or a combination thereof.

In some embodiments, the effective amounts of clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, per unit weight of a subject in the methods above include 10-100 μg, 100-200 μg, 200-300 μg, 300-400 μg, 400-500 μg, 500-600 μg, 600-700 μg, 700-800 μg, 800-900 μg, 1-5 mg, 5-10 mg, 10-20 mg, 20-30 mg, 30-40 mg, 40-50 mg, 50-60 mg, 60-70 mg, 70-80 mg, 80-90 mg, 90-100 mg, 100-200 mg, 200-300 mg, 300-400 mg, 400 mg-500 mg, 500 mg-1 g, or 1 g-10 g. Unit weight of a subject can be per kg of body weight or per subject. In one embodiment, an effective amount of clazakizumab for reducing the level of DSA and desensitization a previously HLA-sensitized human subject in need of or having received an allograft kidney transplant is about 25 mg/month. In one embodiment, an effective amount of clazakizumab for reducing the level of DSA and desensitization a previously HLA-sensitized human subject in need of or having received an allograft kidney transplant is not 25 mg/month.

In further embodiments, the effective amount of clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, may be in the range of 0.01-0.05 mg/kg, 0.05-0.1 mg/kg, 0.1-1 mg/kg, 1-5 mg/kg, 5-10 mg/kg, 10-50 mg/kg, 50-100 mg/kg. In additional embodiments, the effective amount of clazakizumab, an antigen-binding fragment of clazakizumab, or a disclosed polypeptide is about 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, 10-11 mg/kg, 11-12 mg/kg, 12-13 mg/kg, 13-15 mg, 15-20 mg/kg or 20-25 mg/kg. In additional embodiments, the effective amount of the clazakizumab, an antigen-binding fragment of clazakizumab, or a disclosed polypeptide is any one or more of about 100-125 mg, 125-150 mg, 150-175 mg, 160-170 mg, 175-200 mg, 155-165 mg, 160-165 mg, 165-170 mg, 155-170 mg, or combinations thereof, which may be administered over 1, 2, 3, 4, 5, 6, 7, 8, 9, 10, 11, 12, 13, 14, 15, 16, 17, or 18 doses where some are before and others are after transplantation.

In various embodiments, the clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, suitable for administration in the disclosed methods, is administered at any one or more of the dosages described herein at least once 1-7 times per week, 1-7 times per month, or 1-12 times per year, or one or more times as needed, for 1 month, 2 months, 3 months, 4 months, 5 months 6 months, 7 months, 8 months, 9 months, 10 months, 11 months, 12 months, 14 months, 16 months, 18 months, about 24 months, about 30 months, about 36 months or combinations thereof.

Pharmaceutical Composition

In various embodiments, the present invention provides a pharmaceutical composition. The pharmaceutical composition includes (1) clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7, and (2) pharmaceutically acceptable excipients.

The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to amino acids, starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In one embodiment, the disclosed methods involve administering a pharmaceutical composition which includes L-histidine, L-histidine monohydrochloride, sorbitol, polysorbate-80, and water for injection, and clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7.

In various embodiments, the pharmaceutical compositions according to the invention may be formulated for delivery via any route of administration. In one embodiment, the pharmaceutical composition is administered intravenously or subcutaneously to the subject. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion. Pharmaceutically acceptable solid or liquid carriers may be added to enhance or stabilize the composition, to facilitate preparation of the composition, or to provide sustained or controlled release (or increase the half-life) of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. Emulsion carriers include liposomes, or controlled release polymeric nanoparticles known in the art. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

Anti-Infectious Agents

Various embodiments provide that the methods for desensitization further includes administering one or more anti-infectious agents, preferably post-transplantation, as a prophylaxis or therapeutics against bacterial, viral or fungal infections.

Exemplary anti-infectious agents suitable for use in the disclosed methods include antibiotics such as aminoglycosides (e.g., amikacin, gentamicin, kanamycin, neomycin, netilmicin, streptomycin, tobramycin, paromomycin), ansamycins (e.g., geldanamycin, herbimycin), carbacephems (e.g., loracarbef), carbapenems (e.g., ertapenem, doripenem, imipenem, cilastatin, meropenem), cephalosporins (e.g., first generation: cefadroxil, cefazolin, cefalotin or cefalothin, cefalexin; second generation: cefaclor, cefamandole, cefoxitin, cefprozil, cefuroxime; third generation: cefixime, cefdinir, cefditoren, cefoperazone, cefotaxime, cefpodoxime, ceftazidime, ceftibuten, ceftizoxime, ceftriaxone; fourth generation: cefepime; fifth generation: ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin), antibiotic polypeptides (e.g., bacitracin, colistin, polymyxin b), quinolones (e.g., ciprofloxacin, enoxacin, gatifloxacin, levofloxacin, lomefloxacin, moxifloxacin, norfloxacin, ofloxacin, trovafloxacin), rifamycins (e.g., rifampicin or rifampin, rifabutin, rifapentine, rifaximin), sulfonamides (e.g., mafenide, prontosil, sulfacetamide, sulfamethizole, sulfanilamide, sulfasalazine, sulfisoxazole, trimethoprim, trimethoprim-sulfamethoxazole (co-trimoxazole, “tmp-smx”), and tetracyclines (e.g., demeclocycline, doxycycline, minocycline, oxytetracycline, tetracycline) as well as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin combination, and tinidazole. In some embodiments, methods of reducing donor specific antibodies before and/or after allograft transplantation in an HLA-sensitized subject include administering an effective amount of clazakizumab or IL-6 binding, antibody fragment of clazakizumab to the subject, and administering an effective amount of ganciclovir, valganciclovir, fluconazole, trimethoprim, sulfamethoxazole, or a combination thereof to the subject.

Kits

In various embodiments, the present invention provides a kit for desensitization for organ transplant recipients. The kit is an assemblage of materials or components, including clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7; an instruction or manual for administration for desensitization before and after organ transplantation; one or more vessels as containers; and optionally one or more diluents.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or inhibit cancer cachexia in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing clazakizumab, an IL-6 binding fragment of clazakizumab, or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Study: A Phase I/II Trail to Evaluate the Safety and Tolerability of Clazakizumab to Eliminate Donor Specific HLA Antibodies and Improve Transplant Rates in Highly-HLA Sensitized (HS) Patients Awaiting Renal Transplant.

This study is an open label design to assess the safety and efficacy of clazakizumab in eliminating DSAs and inducing Treg subsets in HS patients awaiting HLA incompatible (HLAi) renal transplantation. The protocol is outlined in FIGS. 5 and 6. Safety determinations is aimed at assessments of any side effects associated with clazakizumab administration and risk for infectious complications associated with clazakizumab therapy for desensitization of HS patients awaiting renal HLAi transplantation. Limited efficacy determinations include assessment of the reduction of DSA levels that allow rates of transplantation to increase and subsequent prevention of ABMR (eGFR, SCr) after clazakizumab treatment.

The tested agent, Clazakizumab, has an active ingredient of genetically engineered humanized anti-IL-6 monoclonal antibody. Manufactured by Ajinomoto Althea (San Diego Calif.), it has a strength of 25 mg/mL. It contains excipients including L-histidine, L-histidine monohydrochloride, sorbitol, polysorbate-80, and water for injection. In a single-dose vials of 25 mg/mL for undiluted injection. Clazakizumab vials should be stored at ≤−20° C. (−4° F.) with protection from light. Prepared syringes may be stored for up to 24 hours in a refrigerator, 2°-8° C. (36°-46° F.)≤−20° C. (−4° F.), and up to 4 hours of the 24 hours may be at room temperature, 15°-25° C. (59°-77° F.). The prepared syringes should be protected from light. Prior to administration, clazakizumab should reach room temperature by storing unrefrigerated for 30 to 60 minutes before use.

### Procedures

This is a Phase I/II clinical study of clazakizumab in highly-HLA sensitized patients awaiting renal transplant. The study is intended to have a duration of 3 years. HLA antibodies was detected using solid phase assay systems currently utilized at the Cedars-Sinai Medical Center HLA Laboratory.

Generally, patients entering the study initially received PLEX (5-7 sessions)+IVIG and received clazakizumab 25 mg subcutaneously (SC) one week post-IVIG. If no safety/tolerability/efficacy issues were observed after the initial dose, those patients received 5 additional injections every four weeks (Q4W). If patients receive an HLAi transplant, clazakizumab was continued for 6M post-transplant at 25 mg SC Q4W for 6 doses (starting at Day 5 post-transplant). A protocol biopsy was performed at 6M post-transplant to assess the allograft for evidence of ABMR, including C4d staining and TG using Banff 2015 criteria. Patients would continue another 6 doses over 6 months if improvements are seen after the 6th dose of clazakizumab. Patients who develop evidence of persistent allograft dysfunction may have non-protocol biopsies for cause. Patients who received 12 doses of clazakizumab post-transplant would receive a 12M protocol biopsy. In the event a patient did not show improvement after receiving 6 doses of clazakizumab, no further treatment was given and the patient returned at Day 365 for a final study visit.

Specifically, at transplantation (Day 0), there was one dosage administration of IVIG (IVIG #1). A second dosage of IVIG was administered at Day 1. Subsequently treatment period began. Clazakizumab was administered six times at Day 5±2 d, Day 30±7 d, Day 60±7 d, Day 90±14 d, Day 120±14 d, and Day 150±14 d, respectively.

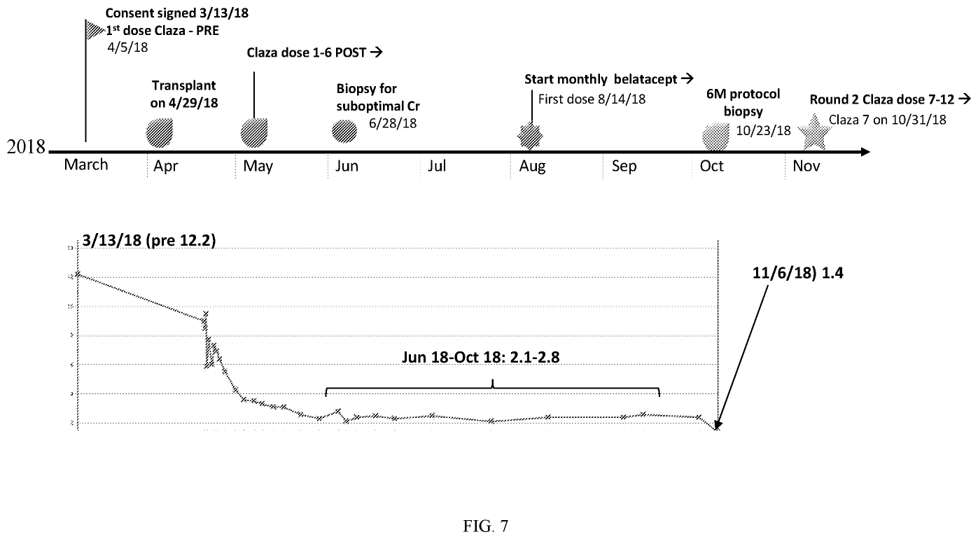

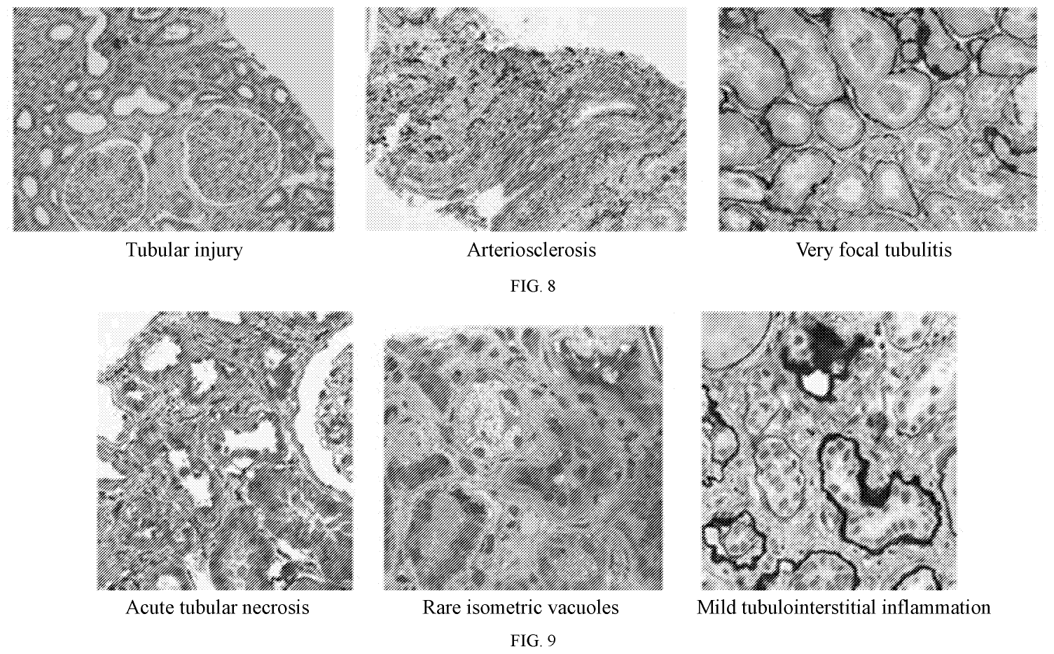

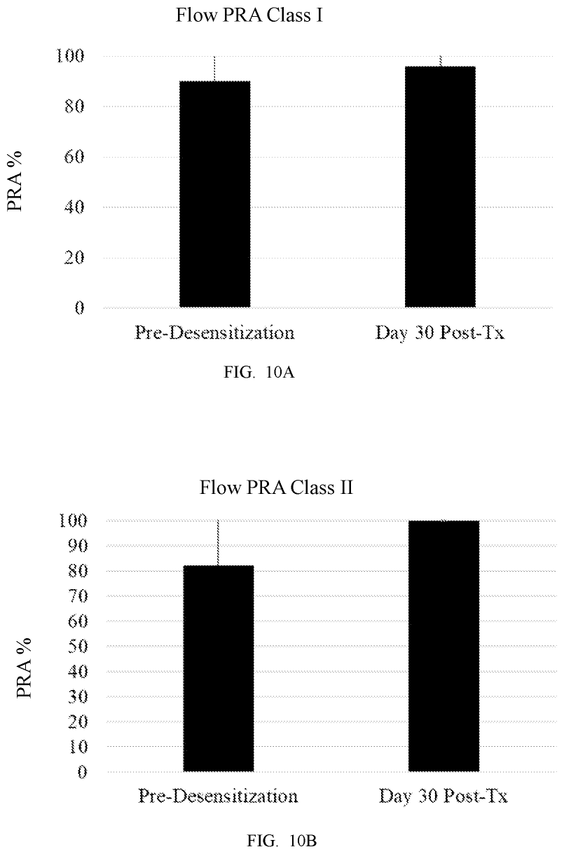

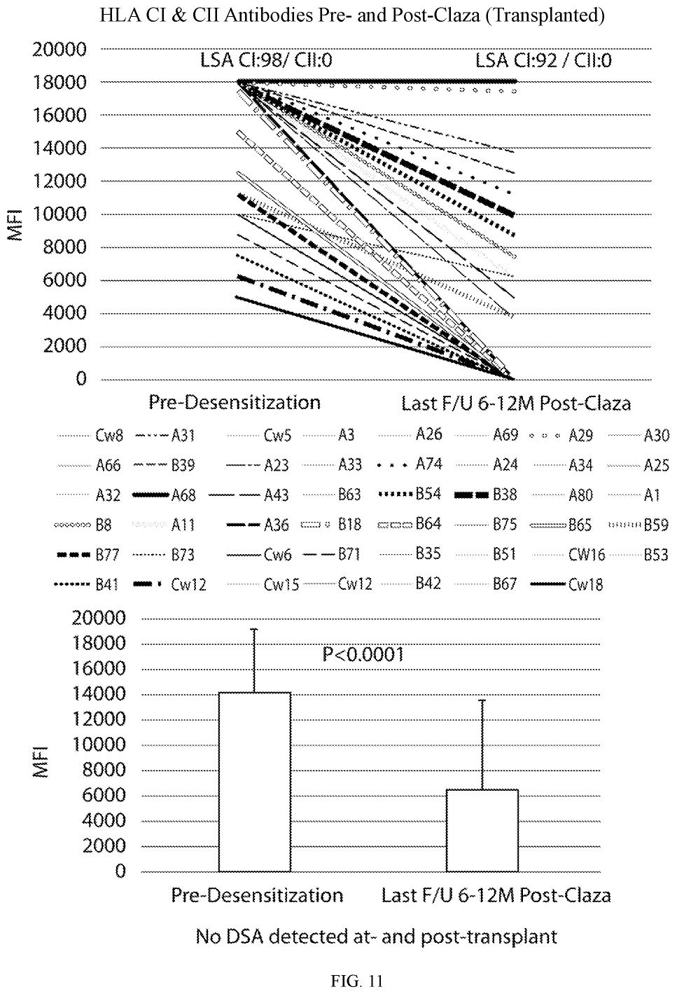

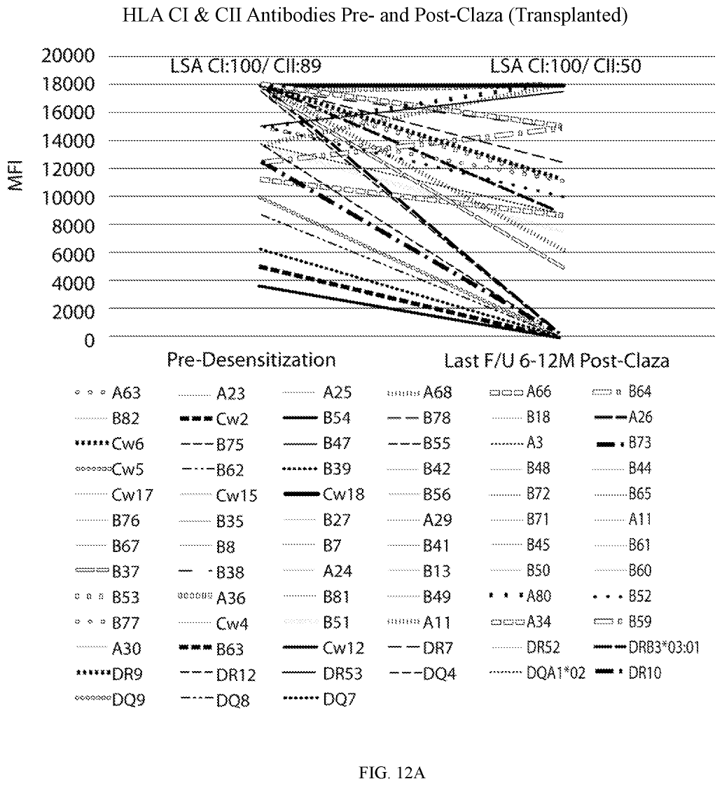

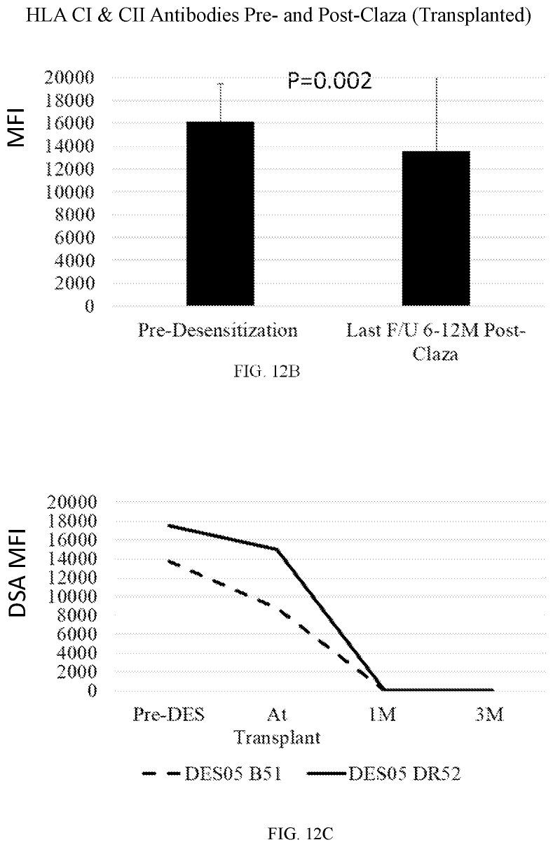

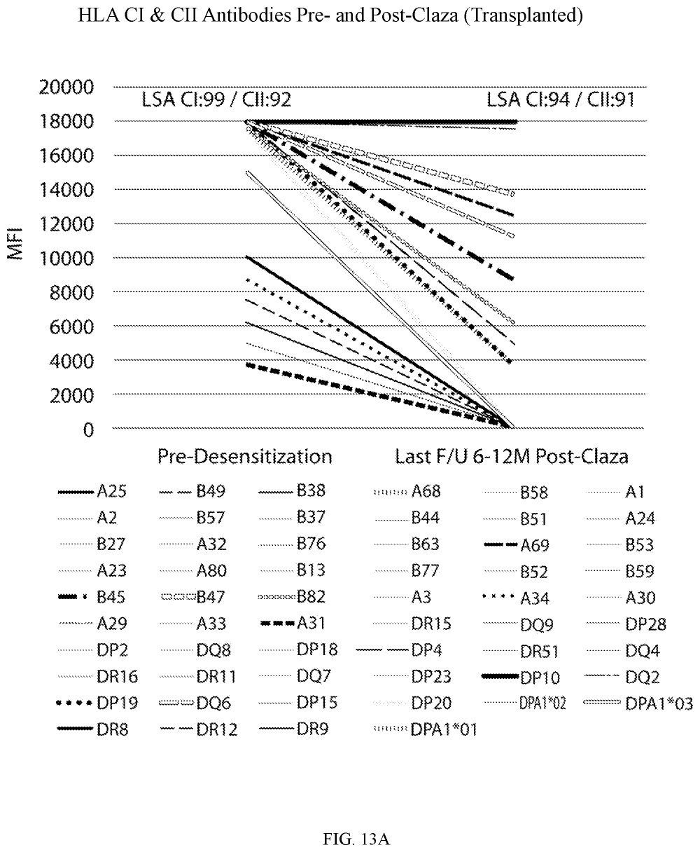

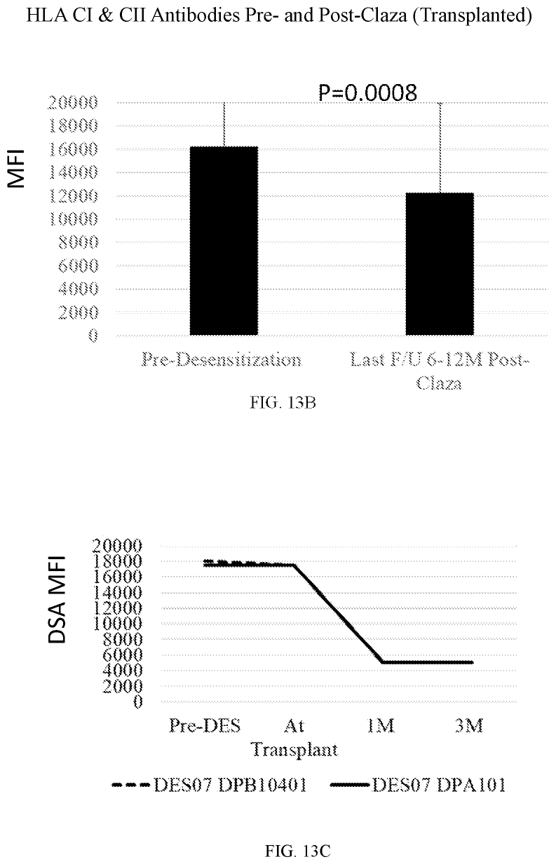

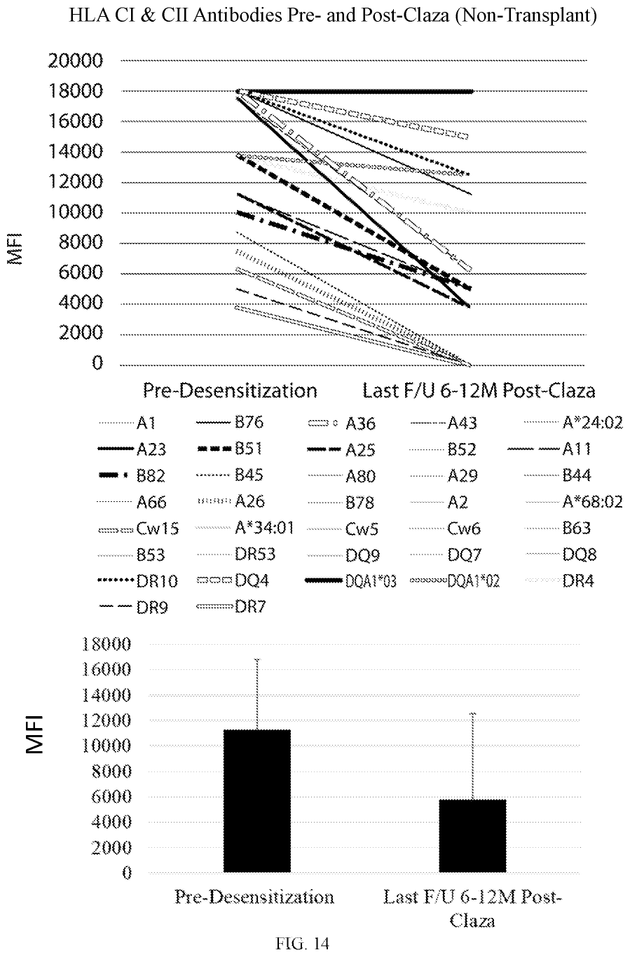

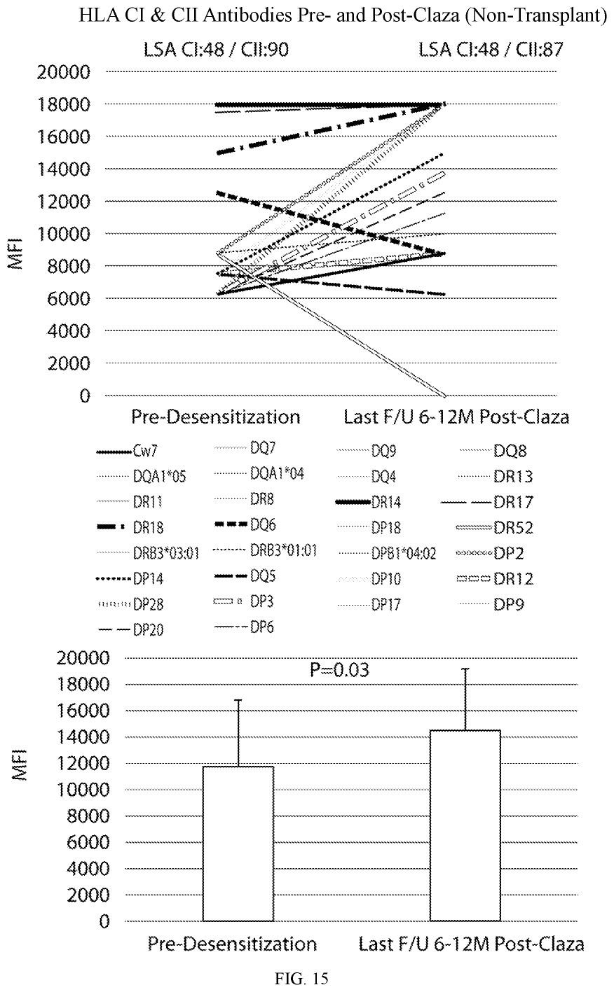

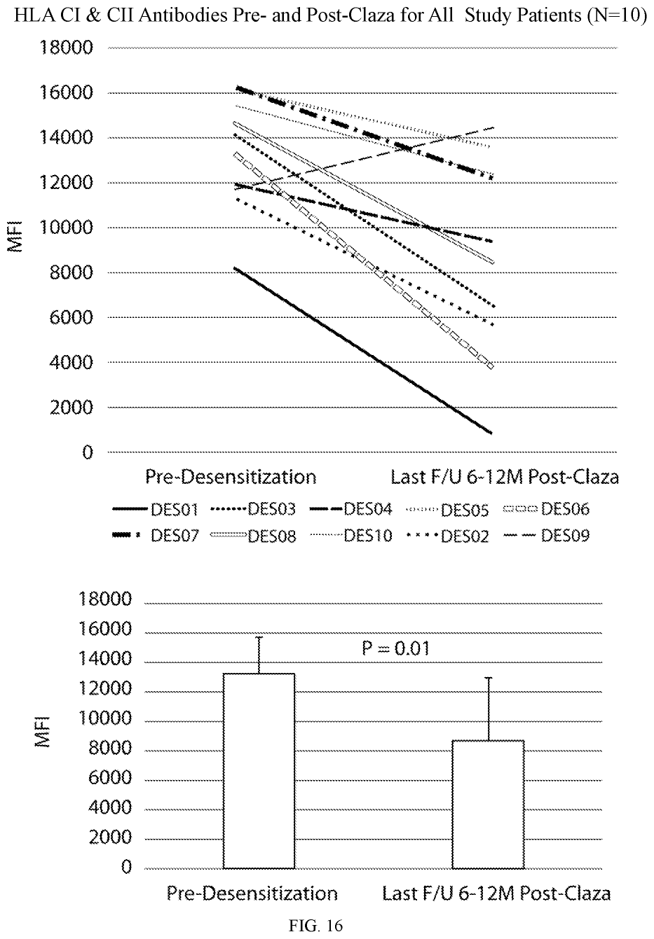

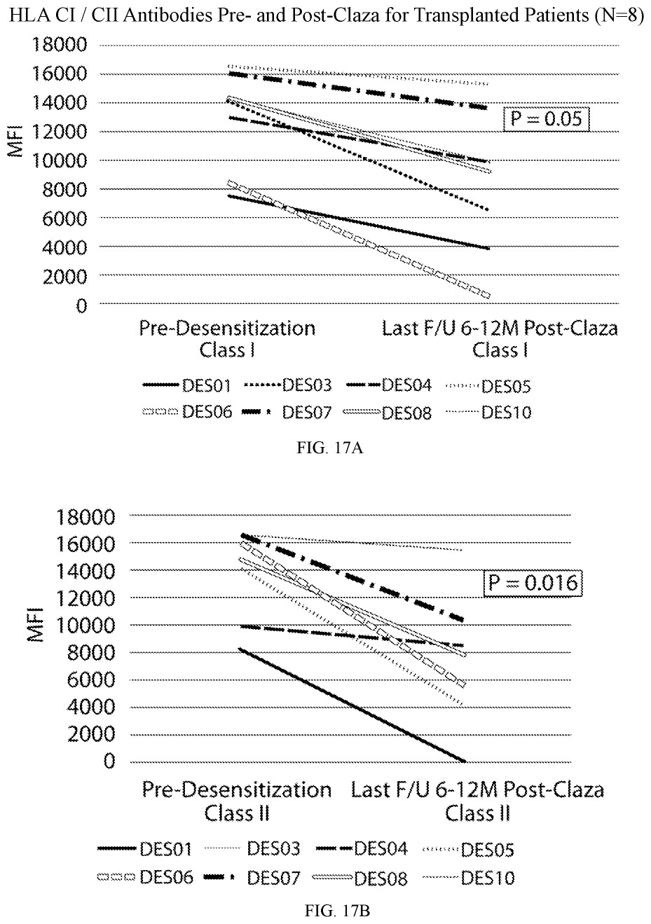

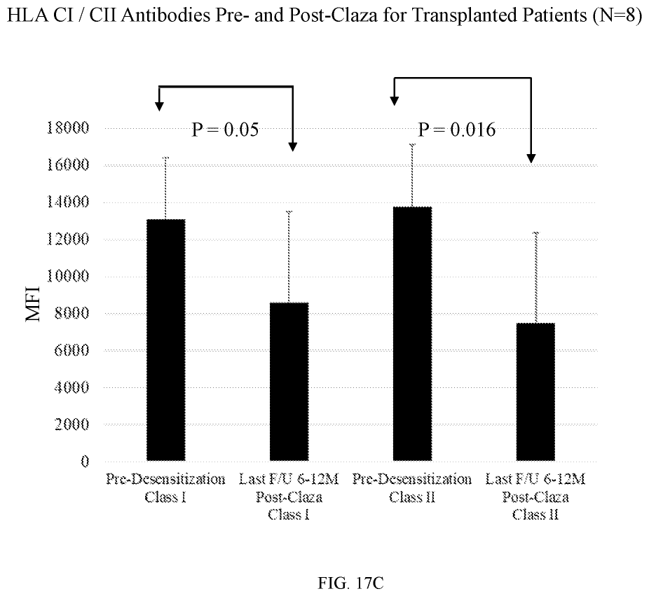

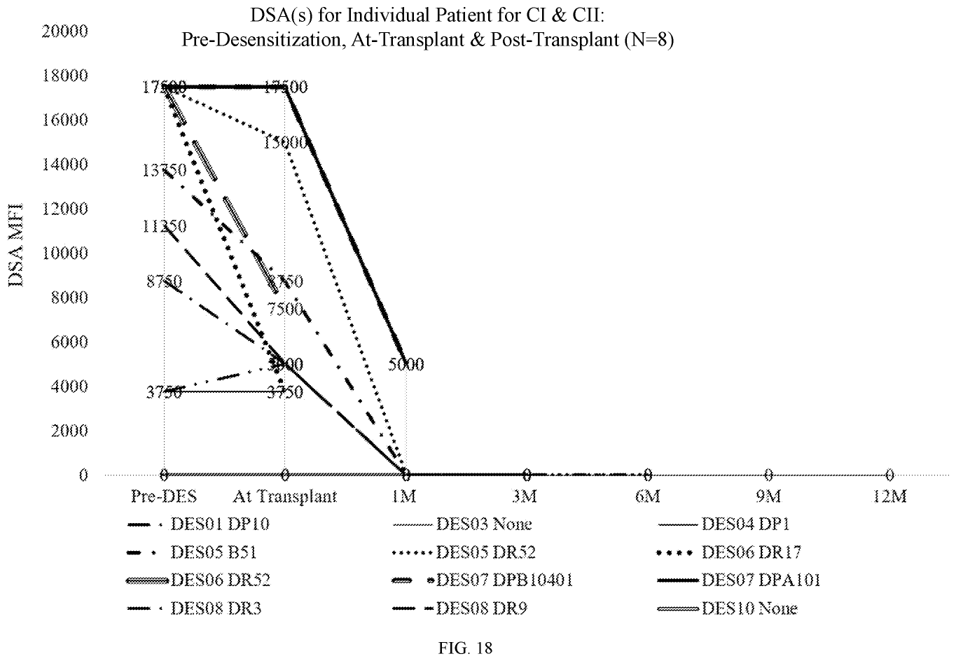

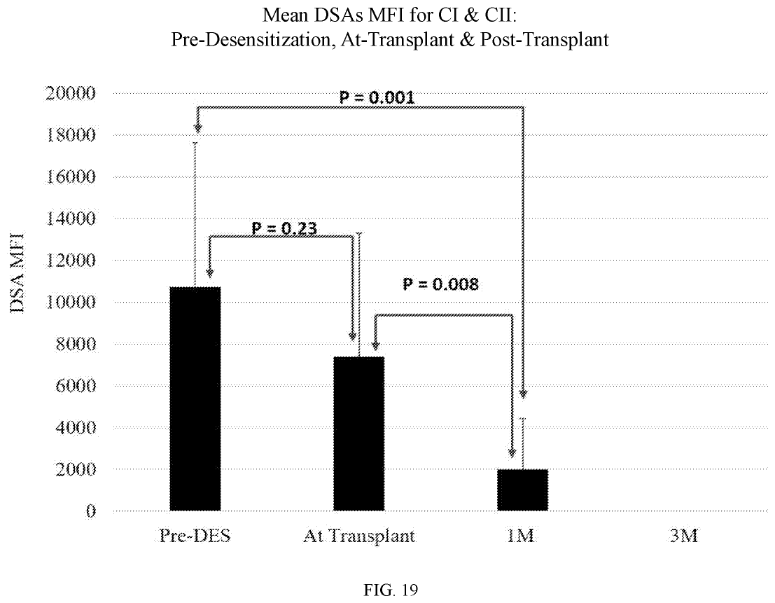

FIG. 7 depicts the timeline of study for the patient ClazaDes03 and his creatinine level over time. Patient “ClazaDES03” is a 32-year-old male with a history of end-stage renal disease secondary to unclear etiology. He is status post living unrelated kidney transplant in 2012 that failed in 2016. For patient “ClazaDES03,” the transplantation took place after the first dose of clazakizumab (25 mg subcutaneously) and before the second dose of clazakizumab. This patient received PLEX (5-7 sessions) on Day −15; IVIG at 2 g/kg on Day 0; Pre-transplant #1 clazakizumab (25 mg SQ) on Day 7 (on Apr. 5, 2018); underwent deceased donor kidney transplant on Day 21 (on Apr. 29, 2018); Post-transplant #1-#6 clazakizumab (25 mg SQ) at about monthly intervals; induction with alemtuzumab (CAMPATH 1H), and maintained on tacrolimus, mycophenolate mofetil (MMF) and prednisone. When the renal allograft biopsy was examined on Jun. 28, 2018 (FIG. 8) about two months after transplantation and showed suboptimal creatinine level, monthly dosing of belatacept began on Aug. 14, 2018. On Oct. 23, 2018, the 6M biopsy was examined (FIG. 9). Starting on Oct. 31, 2018, a second round (#7-#12) of clazakizumab dosing began (#7 Post-transplant clazakizumab). There were no donor-specific antibodies present since transplantation for this patient.

Anti-HLA antibodies may result naturally or from previous pregnancy, transfusions, or prior transplants. Patients treated with clazakizumab×6 doses for desensitization had blood sampling for HLA antibodies, and other monitoring blood samples as well as immunologic studies. If patients received an HLAi transplant during the study, they would receive the standard post-transplant immunosuppressive protocol, and clazakizumab 25 mg subcutaneously every four weeks for 6 doses with immune monitoring. Immune monitoring in blood samples includes for Treg, Tfh, Th17 and B-cell subsets as well as IL-6 and CRP monitoring, which was carried out at the Cedars-Sinai Transplant Immunology Laboratory

### Results

FIG. 1 shows the DSA profile in the study for subject “ClazaDES01,” who is a 50-year old African American female with a history of end-stage renal disease (ESRD) secondary to biopsy proven focal segmental glomerulosclerosis (FSGS) and who had been on dialysis since November 2008 (i.e., approximately 10 years' of wait-time for B+ blood type) with calculated panel reactive antibodies (cPRA) of 58%. Patient's sensitizing events included pregnancies ×4 and blood transfusion.

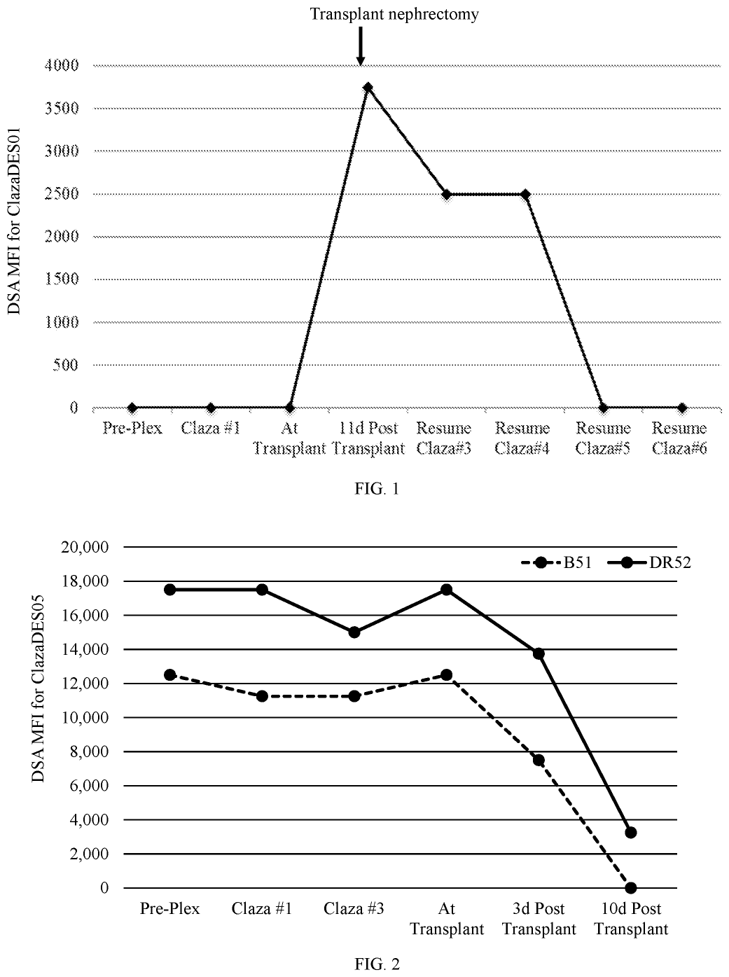

FIG. 2 shows the DSA profile for subject “ClazaDES05” pre- and post-transplantation. (Median fluorescence intensity, MFI). Subject “ClazaDES05” is 36-year old female with a history of ESRD secondary to IgA nephropathy, and she had been on dialysis since June 2008 (i.e., approximately 10 years' of wait-time for A+ blood type), with cPRA 100%. Patient's sensitizing event included previous transplant, and blood transfusion. Subject “ClazaDES05” received a deceased donor kidney transplant after 4 doses of clazakizumab. Patient had 2 DSAs pre- and post-transplant (Class I and II). DSA strengths for class I were reduced from MFI>12,500 MFI at transplantation to MFI=0 at 10 days post-transplantation, and for class II from MFI>17,500 at transplantation to MFI>3250 at 10 days post-transplantation. Patient continued with monthly clazakizumab for 6 months post-transplantation as per study protocol.

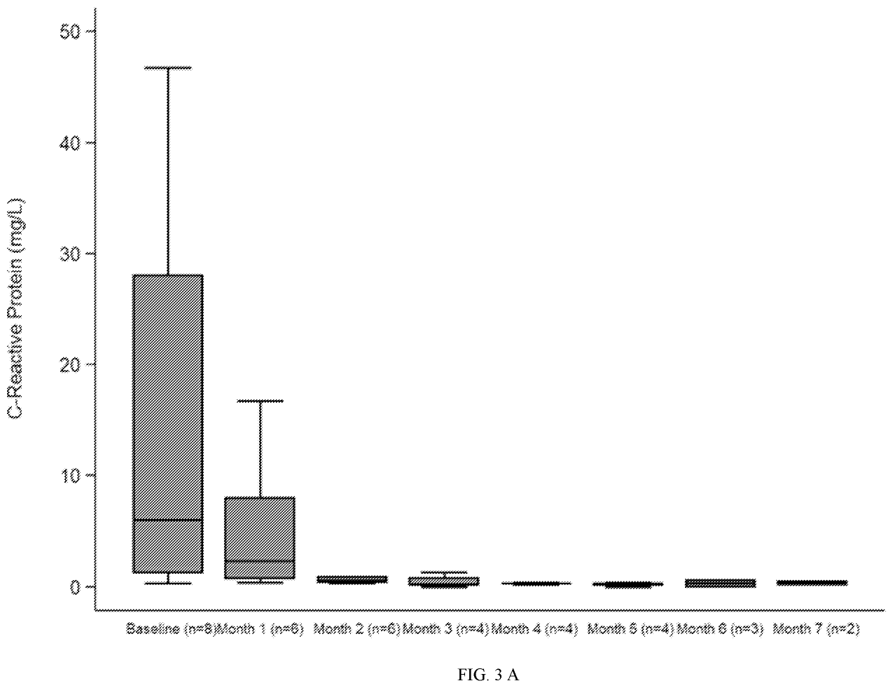

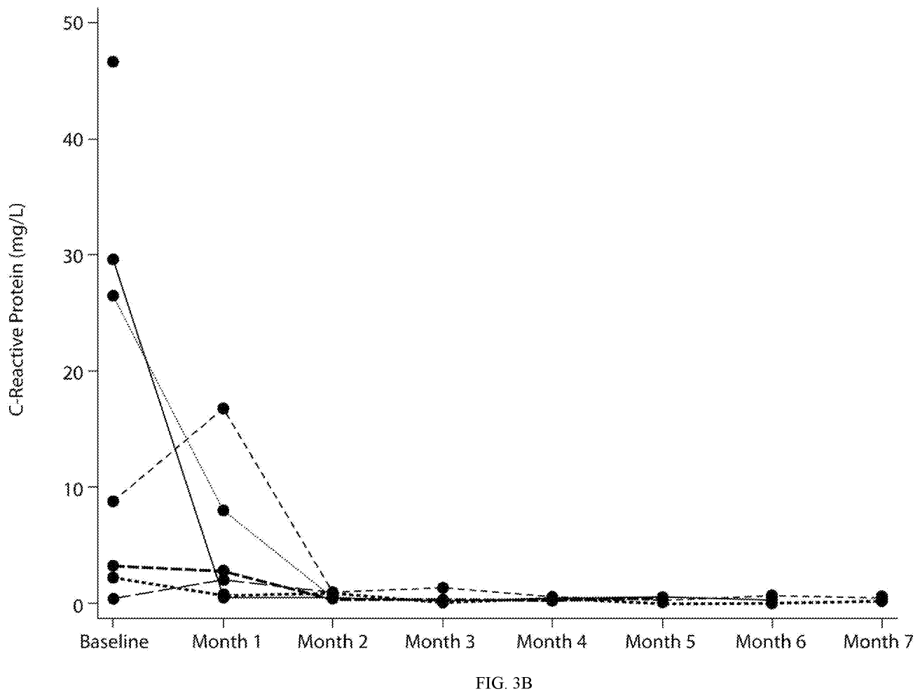

FIG. 3A shows the overall C-reactive protein amount in the clazakizumab desensitization study. Overall, C-reactive protein (CRP) was reduced from baseline to nearly zero by the second month. Number of subjects included in the analysis for each time point is indicated in parentheses. FIG. 3B shows the individual C-reactive protein amounts in the clazakizumab desensitization study from baseline to the seventh month.

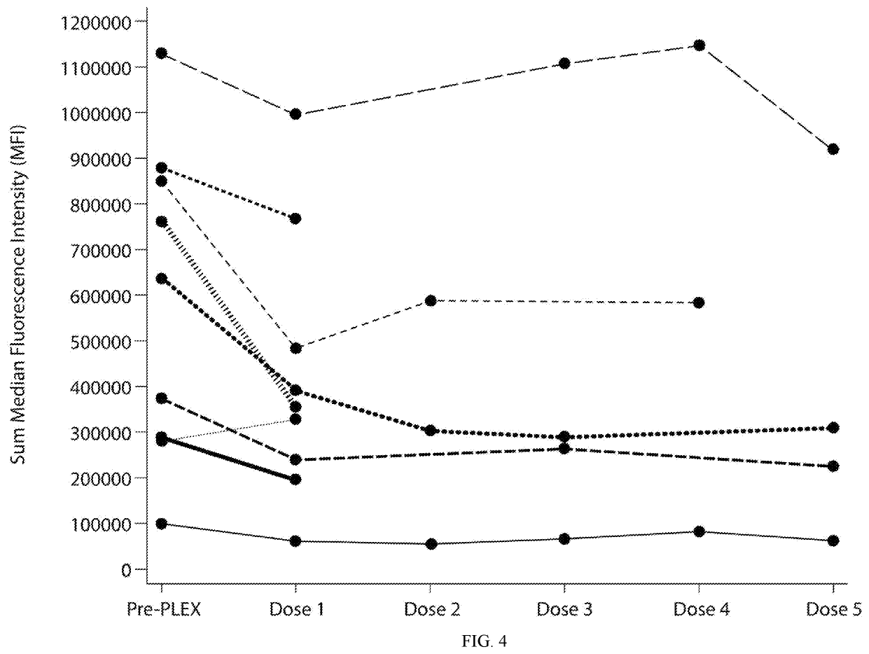

FIG. 4 shows the sum of MFI over time from before plasmapheresis (PLEX) (pre-PLEX) to the fifth dose of clazakizumab (N=9). Typically, MFI tends to rebound by approximately 1-3 months after completion of PLEX/IVIG. Here, with monthly clazakizumab injection, the sum of MFI remained reduced over time when compared to pre-PLEX. Three patients were transplanted to date. Patients ClazaDES01 and ClazaDES03 were transplanted after the first dose of clazakizumab. Patient ClazaDES05 received a transplant after the fourth dose of clazakizumab.

For patient “ClazaDES03,” FIG. 7 shows his creatinine (mg/dL) level over time, comparing before the desensitization treatment and after the desensitization treatment and kidney transplantation. A low level of creatinine was maintained following transplantation with the two rounds of post-transplant clazakizumab administration. FIG. 8 shows the 2-month renal transplant biopsy of patient “ClazaDES03.” In this biopsy, there was a mild acute tubular injury; a mild-to-moderate arterio- and mild arteriolosclerosis, which was consistent with donor disease; no diagnostic evidence of acute rejection (at most borderline for cell-mediated rejection by Banff criteria; and mesangial IgA deposits without associated proliferative glomerular changes. Because there was no IgA staining on biopsies from the patient's previous renal transplant in 2012, it was believed that the IgA present on this biopsy was likely to be donor-related. FIG. 9 shows the 6-month renal transplant biopsy of patient “ClazaDES03.” In this biopsy, there was acute tubular necrosis with rare isometric epithelial vacuoles; mild tubulointerstitial inflammation (at most borderline changes for cell-mediated rejection); arteriosclersosis; and minimal interstitial fibrosis/tubular atrophy. Rarely isometric epithelial vacuoles are detected and it may be related to acute calcineurin inhibitor toxicity of recent IVIG therapy. There was no diagnostic features of antibody-mediated rejection or polyomavirus nephropathy.

Overall, the desensitization treatment using clazakizumab reduced HLA antibodies and admitted of transplantation of at least 40% of patients in the study.

A total of ten patients were enrolled from March to November 2018. Nine patients were admitted of transplantation: eight were transplanted during the study period and the ninth patient was transplanted two months after completion of the study. Four patients have reached 12-month study period. All infusions received were well tolerated. There was no graft loss or patient death observed and no significant infections attributed to clazakizumab or requiring discontinuation of clazakizumab. Renal function at six months was stable. The demographics and immunologic/transplant characteristics are summarized in Tables 1 and 2.

TABLE 1
Demographics and baseline characteristics
of nine subjects receiving transplantation.

| Patient Demographics | Transplanted N = 9 |
|---|---|
| Gender (Male) (No., %) | 5 (55.6%) |
| Age Range (Yrs) | 32-66 |
| ESRD (No., %) | |
| HTN | 1 (11.1%) |
| Glomerulonephritis | 3 (33.3%) |
| IgA Nephropathy | 2 (22.2%) |
| Unclear Etiology | 1 (11.1%) |
| Reflux Nephropathy | 1 (11.1%) |
| PKD | 1 (11.1%) |
| African American (No., %) | 3 (33.3%) |
| Cold Ischemia Time (Hrs) | 19.3 ± 7.15 |
| Delayed Graft Function (No., %) | 6 (66.7%) |
| Time from Dialysis to Transplant (D) | 3095 ± 1545.18 |
| Mean Time to Transplant from last Claza (D) | 143.6 ± 91.5 |

TABLE 2
Immunologic and transplant characteristics of
the nine subjects receiving transplantation.

| Immunologic/Transplant Characteristics | Transplanted N = 8 | |
|---|---|---|
| Previous Tx (No., %) | 9 | (100%) |
| cPRA (No., %) | | |
| 50-60% | 1 | (11.1%) |
| 90-98% | 1 | (11.1%) |
| 99-100%\hat{ } (range 99.51-99.93) | 7 | (77.8%) |
| HLA A/B/DR mismatch, mean (SD) | 1.67 ± 1.96 | |
| Flow CMX Positivity (cut off: | | |
| T <70 MCS pronase; B <130 MCS pronase) | | |
| B-cell only | 6 | (66.7%) |
| Both T-cell + B-cell | 1 | (11.1%) |
| None | 2 | (22.2%) |
| DSA at transplantation | | |
| Class II only | 6 | (66.7%) |
| Both Class I + Class II | 1 | (11.1%) |
| None | 2 | (22.2%) |
| Graft loss (No., %) | | |
| Surgical/Technical | 1 | (11.1%) |
| Death (No., %) | 0 | (0%) |
| Viral Infections | | |
| CMV Viremia | 0 | (0%) |
| BK Viremia | 1 | (11.1%) |
| eGFR mean (SD) (ml/min/1.73 m²) | | |

| | |
|---|---|
| At 3 months | 56.8 ± 27.8 |

\hat{ }One patient received 0 MM

All except for the ninth patient had undergone 6-month protocol biopsy. Two patients (25%) have biopsy proven rejection: 1 patient with chronic active cell mediated rejection, Banff grade 1A; 1 patient with chronic active antibody mediated rejection and cell mediated rejection, Banff grade 1B. Both patients responded to treatment as per the study center's standard-of-care protocol.

Seven (78%) of nine transplanted patients had DSA pre-desensitization and at the time of transplantation. Only 3 patients (33%) had DSAs detected at one month; 2 patients (22%) had DSAs detected at three months; and no patients had DSA detected at six months (6M), nine months (9M) and 12 months (12M) (including those who had DSAs detected at one month and three months). The DSA MFI values (mean±SD) for pre-desensitization, at the time of transplantation, at one month and three months were as follows: 11060±6990, 7980±6260, 1923±3973, 1040±2650; and 0±0 for 6M, 9M and 12M respectively. The mean DSA MFI was significantly reduced when compared Pre-desensitization vs. at 1M (p=0.0004) and at 3M (p=0.0001) and at transplant vs. 1M (p=0.007) and at 3M (p=0.001).

There were seven SAEs, but all felt unrelated to clazakizumab. These SAEs included wound dehiscence requiring wound re-closure (1 SAE), hematuria and UTI (1 SAE), and bacteremia (1 SAE) prior to receiving 1st dose of the study drug, thrombosis of right external iliac artery with graft loss (1 SAE), persistent surgical site pain requiring CT guided drainage of peri-transplant fluid which grew MSSA (1 SAE), biopsy proven chronic active ABMR as a result of delayed in clazakizumab administration d/t infection and chronic thrombocytopenia (1 SAE), perinephric fluid collection requiring CT guided drainage (1 SAE).

### Major Secondary Objectives

To determine if clazakizumab treatment, can significantly reduce or eliminate ABMR episodes and C4d deposition in incompatible allografts transplanted to highly-HLA sensitized patients post-clazakizumab desensitization. Assess allograft function up to 6-12 months (6-12 M) post-transplant, determine renal function using serum creatinine (SCr), Modification of Diet in Renal Disease (MDRD) GFR calculations (Schwartz equation will be used to estimate creatinine clearance (CrCl) for patients under 18 years of age) and DSA levels. A protocol biopsy was performed at 6M post-Clazakizumab therapy. In addition, several immunologic determinations of blood samples were assessed at time points before and after initiation of clazakizumab therapy. These include the following:

Assessment of T_{reg} cells (CD4+, CD25+, FoxP3+CD127^{dim})

Assessment of T_{fh} cells (CD4+, ICOS+, CXCR5+, IL-21+)

Assessment of circulating plasmablast (CD19+, CD38+, CD27+, IL-6+)

Assessment of CRP and IL-6levels

These secondary end points would help us understand the biology of alloimmune responses to allografts and to determine the ability and mechanisms of clazakizumab's beneficial effects. Viral PCRs were monitored as per standard-of-care.

### Inclusion Criteria

Age 15-75 years at the time of screening. HS patients (cPRA≥50%) awaiting DD or LD kidney transplant on the UNOS list. Previous history of pregnancies, blood transfusion and/or renal transplant. Subject/Parent/Guardian must be willing to participate fully with study requirements. Subject/Parent/Guardian must be able to understand and provide informed consent. Pneumococcal vaccinated. Negative Tuberculin (ppd) placement result or negative Quantiferon TB gold results. These individuals must also have sufficient wait time on the LINOS list to allow for frequent offers with a history of positive crossmatches (DD) or an incompatible (LD) with a positive flow cytometry (FCMX) and negative complement-dependent cytotoxicity (CDC+) crossmatch. Patients proceeding to HLAi transplant after desensitization would have a CDC CMX negative at 1:2 dilution, FCMX<225 channel shifts and DSAs that are at an acceptable MFI as was previously defined.

### Exclusion Criteria

Multi-organ transplant (e.g. kidney and pancreas).
Intolerability to clazakizumab or other IL-6 inhibitor therapies.
Lactating or pregnant females.
Women of child-bearing age and male partners of women of child-bearing age who are not willing or able to practice FDA-approved forms of contraception during study and for 5 months after last dose.
HIV-positive subjects.
Subjects who test positive for HBV by HBVeAg/DNA or HCV infection [positive Anti-HCV (EIA) and confirmatory HCV RIBA].
Subjects with latent or active TB. Subjects must have negative Quantiferon TB gold test result.
Recent recipients of any licensed or investigational live attenuated vaccine(s) within two months of the screening visit (including but not limited to any of the following: Adenovirus [Adenovirus vaccine live oral type 7], Varicella [Varivax], Hepatitis A [VAQTA], Rotavirus [Rotashield], Yellow fever [Y—F-Vax], Measles and mumps [Measles and mumps virus vaccine live], Measles, mumps, and rubella vaccine [M-M-R-II], Sabin oral polio vaccine, and Rabies vaccines [IMOVAX Rabies I.D., RabAvert]).
A significantly abnormal general serum screening lab result defined as a ANC<2000, platelet count<100×10³/ml, an SGOT or SGPT>1.5× upper limit normal.
Individuals deemed unable to comply with the protocol.
Subjects with active CMV or EBV infection as defined by CMV-specific serology (IgG or IgM) and confirmed by quantitative PCR with or without a compatible illness (Quantitative PCR cut off defined as having >50 copies of CMV or EBV DNA/PCR) Use of investigational agents within 4 weeks of participation.
History or active Inflammatory Bowel Disease or Diverticular Disease or gastrointestinal perforation
Recent infection (within past 6 weeks of screening) requiring any antibiotic use (oral, parenteral or topical).
Present or previous (within 5 years) malignancy except for basal cell carcinoma, fully excised squamous cell carcinoma of the skin or non-recurrent (within 5 years) cervical carcinoma-in-situ.

Applicant has also performed a study assessing the cost/benefit analysis of desensitization compared with dialysis. The costs associated with transplantation after desensitization including all medications, organ acquisition, treating rejection episodes, and cost of returning to dialysis for those who lost their allografts compared favorably with the costs of remaining on dialysis over the same period of time. Most important was the survival benefit engendered by transplantation in this cohort. At 3 years, the desensitized and transplanted patients had a mortality rate of 3.5% compared to 22.8% for those remaining on dialysis.

If rejection episodes occur (biopsy-proven) during the study, patients are treated with “pulse” methylprednisolone (10 mg/kg/day, max 1000 mg for >100 kg for 3 days) and anti-thymocyte globulin (1.5 mg/kg daily×4) for cell-mediated rejection episodes that are unresponsive to pulse steroids. Patients experiencing recurrent antibody mediated rejection (ABMR) episodes after study drug treatment will initially receive pulse methylprednisolone (10 mg/kg/day, max 1000 mg for >100 kg) IV daily×3 doses then, depending on severity, IVIG 10% solution 2 gm/kg (max 140 g for >70 kg) IV×1 dose followed by rituximab (375 mg/m²) IV×1 dose. In cases where rapid deterioration of allograft function is seen and/or thrombotic microangiopathy diagnosed, the patient will receive plasma exchange for 3-5 sessions followed by anti-CS (Eculizumab®) IV weekly for 4 weeks (1200 mg week #1 followed by 900 mg/weekly for 3 additional weeks). Efficacy of therapy will be assessed by determining renal functional improvement, monitoring DSA responses and repeat allograft biopsies, if needed. For purposes of this investigation, ABMR is defined as follows: Deterioration of allograft function in a high-risk transplant recipient (i.e. sensitized patient with history of DSAs) measured by serum Cr and eGFR (defined as a decline >30% from baseline); Association with the presence of DSA (usually increasing in strength) measured by LUMINEX techniques; Biopsy evidence based on BANFF 2015 grading which includes: capillaritis, inflammation and C4d deposition.

Adverse events (AEs) and serious adverse events was monitored post treatment with clazakizumab. These included careful attention to infectious complications potentially associated with clazakizumab therapy. Infectious complications associated with IVIG+rituximab desensitization and alemtuzumab induction therapy followed by maintenance therapy with tacrolimus, MMF and prednisone have been assessed by our group. The use of this desensitization protocol followed with alemtuzumab induction does not increase the risk for common or serious infections post-transplant compared to a low risk group of patients. Serious infections were defined as any viral infection and fungal or bacterial infections requiring i.v. antibiotics or hospitalizations. Thus risk for infections in the study group (clazakizumab) after ABMR treatment will likely be similar and comparable to non-sensitized patients. All patients entered into this study are required to be vaccinated for Streptococcus pneumoniae.

In this study, all study patients, regardless of their cytomegalovirus (CMV) status, received IV ganciclovir while inpatients and valganciclovir as outpatients for 6 months post kidney transplant, with dose adjustments for renal function. Fungal prophylaxis was accomplished with fluconazole 100 mg daily for 1 month post-transplant. Pneumocystis jirovecii pneumonia and bacterial prophylaxis is accomplished with trimethoprim 80 mg and sulfamethoxazole 400 mg daily for 12 months post-transplant. Viral polymerase chain reaction assays for CMV, Epstein Barr virus, Parvovirus B-19, Polyoma virus BK and JC were performed on study patients monthly for 6 months post-transplantation.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless otherwise indicated, all numbers expressing quantities should be understood as modified in all instances by the term “about.” The term “about” can refer to ±10% of the value being referred to. If specifically defined and provided for in the claim, the term “about” can refer to ±9%, ±8%, ±7%, ±6%, ±5%, ±4%, ±3%, ±2%, or ±1%) of the value being referred to; for example a claim may state that the value is about X, wherein about is ±6%.

Where a range of values is provided, each numerical value between and including the upper and lower limits of the range is contemplated as disclosed herein. It should be understood that any numerical range recited herein is intended to include all sub-ranges subsumed therein. For example, a range of “1 to 10” is intended to include all sub-ranges between and including the recited minimum value of 1 and the recited maximum value of 10; that is, having a minimum value equal to or greater than 1 and a maximum value of equal to or less than 10. Because the disclosed numerical ranges are continuous, they include every value between the minimum and maximum values.

1. A method for reducing and/or eliminating donor-specific antibody in a human leukocyte antigen (HLA)-sensitized subject, comprising:
administering to the subject an effective amount of clazakizumab; an interleukin-6 (IL-6) binding fragment of clazakizumab; or a polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7,
wherein the subject is in need of or has undergone a solid organ transplantation.

2. The method of claim 1, comprising:
administering to the subject an effective amount of a pharmaceutical composition comprising
the clazakizumab; the IL-6 binding fragment of clazakizumab; or the polypeptide having V_{H} polypeptide containing CDR1, CDR2, and CDR3 polypeptides which respectively are contained in SEQ ID NO: 1, 2 or 3, and 4 and having V_{L} polypeptide containing CDR1, CDR2, and CDR3 polypeptides which respectively are contained in SEQ ID NO: 5, 6, and 7; and
one or more pharmaceutically acceptable excipients.

3. The method of claim 1 or 2, wherein the clazakizumab, the IL-6 binding fragment of clazakizumab, or the polypeptide is administered before the solid organ transplantation.

4. The method of claim 1 or 2, wherein the clazakizumab, the IL-6 binding fragment of clazakizumab, or the polypeptide is administered after the solid organ transplantation, during the solid organ transplantation, or both.

5. The method of claim 1 or 2, wherein the clazakizumab, the IL-6 binding fragment of clazakizumab, or the polypeptide is administered both before and after the solid organ transplantation.

6. The method of claim 1 or 2, further comprising administering a standard-of-care treatment which comprises intravenous immunoglobulin (IVIG) administration, rituximab administration, plasmapheresis, or a combination thereof.

7. The method of claim 6, wherein the standard-of-care treatment is administered before the clazakizumab, the IL-6 binding fragment of clazakizumab, or the polypeptide.

8. The method of claim 1 or 2, wherein the solid organ is a kidney.

9. The method of claim 1 or 2, wherein the solid organ is one or more of heart, liver, lung, pancreas, and intestine.

10. The method of claim 1 or 2, wherein the clazakizumab, the IL-6 binding fragment of clazakizumab, or the polypeptide is administered subcutaneously or intravenously.

11. The method of claim 1 or 2, wherein the clazakizumab, the IL-6 binding fragment of clazakizumab, or the polypeptide is administered subcutaneously at an average dose of about 0.1-1 mg/month, 1-5 mg/month, 5-10 mg/month, 10-20 mg/month, 20-30 mg/month, or 30-40 mg/month for at least one month and up to 18 months.

12. The method of claim 1 or 2, wherein a plurality of doses of the clazakizumab, the IL-6 binding fragment of clazakizumab, or the polypeptide is administered at about monthly intervals for 1 month, 2 months, 3 months, 4 months, 5 months, 6 months, 7 months, 8 months, 9 months, 10 months, 11 months, or 12 months.

13. The method of claim 1 or 2, wherein the clazakizumab, the IL-6 binding fragment of clazakizumab, or the polypeptide is administered subcutaneously at an average dose of about 10-30 mg/time for 1, 2, 3, 4, 5 or 6 times prior to the solid organ transplantation and for 1, 2, 3, 4, 5, 6, 7, 8, 9, 10, 11, or 12 times after the solid organ transplantation.

14. The method of claim 1 or 2, wherein the subject is a human.

15. The method of claim 1 or 2, further comprising administering one or more anti-infectious agents to the subject.

16. The method of claim 15, wherein the one or more anti-infectious agents are administered along with or after the solid organ transplantation.

17. The method of claim 14, wherein the anti-infectious agent comprises ganciclovir, valganciclovir, fluconazole, trimethoprim, sulfamethoxazole, or a combination thereof.

18. The method of claim 1 or 2, further comprising administering a standard-of-care treatment which comprises intravenous immunoglobulin (IVIG) administration, rituximab administration, plasmapheresis, or a combination thereof; an anti-infectious agent; or a combination of the standard-of-care treatment and the anti-infectious agent.

19. The method of claim 1 or 2, further comprising selecting a human subject having calculated panel reactive antibodies (cPRA) of 50% or greater, or performing a panel reactive antibody assay and determining the human subject having cPRA of 50% or greater.

20. The method of claim 1 or 2, wherein after the solid organ transplantation, the subject does not show detectable evidence of developing antibody-mediated rejection of the solid organ transplant, does not show detectable evidence of developing a viral infection, or both.

21. The method of claim 1 or 2, further comprising following the solid organ transplantation, administering an antibody induction therapy comprising alemtuzumab, an anti-thymocyte globulin, or both; administering an immunosuppression therapy comprising tacrolimus, mycophenolate mofetil, prednisone or a combination thereof; or administering an antibody induction therapy and an immunosuppression therapy.

22. The method of claim 1 or 2, wherein the subject in need of the solid organ transplantation undergoes the solid organ transplantation, or the subject has undergone the solid organ transplantation, and the method further comprising conducting one or more times of immune monitoring to the subject comprising assaying a blood sample of the subject to quantify levels of markers comprising CRP, Treg, Tfh, Th17, B-cell, IL-6, plasma cells, plasmablast IgG, or a combination thereof.

23. The method of claim 22, when the immune monitoring indicates an improvement based on one or more decreased levels of the markers compared to a baseline measurement taken at or before the solid organ transplantation or based on one or more decreased levels compared to those obtained from a previous immune monitoring, further administration of clazakizumab, the IL-6 binding fragment of clazakizumab or the polypeptide is discontinued or limited to no more than additional 6 months; when the immune monitoring indicates poor performance based on comparable or increased levels of the markers compared to the baseline measurement or to those obtained from a previous immune monitoring, one or more doses of the clazakizumab, the IL-6 binding fragment of clazakizumab or the polypeptide is administered.

24. The method of claim 1 or 2, wherein the subject in need of the solid organ transplantation undergoes the solid organ transplantation, or the subject has undergone the solid organ transplantation, and the method further comprising measuring the amount of glomerular filtration rate, DSA, or both, after the solid organ transplantation.

25. The method of claim 24, when the amount of glomerular filtration rate, DSA, or both is similar or reduced compared to a baseline level measured before or at the solid organ transplantation, further administration of clazakizumab, the IL-6 binding fragment of clazakizumab or the polypeptide is discontinued or limited to no more than additional 6 months; when the amount of glomerular filtration rate, DSA, or both is higher than the baseline level, one or more doses of the clazakizumab, the IL-6 binding fragment of clazakizumab or the polypeptide is administered.


SEQUENCE LISTING (12 sequences)

SEQ ID NO: 1 - protein, 5 aa, Artificial Sequence
  VH CDR1
NYYVT

SEQ ID NO: 2 - protein, 16 aa, Artificial Sequence
  VH CDR2
IIYGSDETAYATWAIG

SEQ ID NO: 3 - protein, 16 aa, Artificial Sequence
  Another VH CDR2
IIYGSDETAYATSAIG

SEQ ID NO: 4 - protein, 12 aa, Artificial Sequence
  VH CDR3
DDSSDWDAKFNL

SEQ ID NO: 5 - protein, 11 aa, Artificial Sequence
  VL CDR1
QASQSINNELS

SEQ ID NO: 6 - protein, 7 aa, Artificial Sequence
  VL CDR2
RASTLAS

SEQ ID NO: 7 - protein, 12 aa, Artificial Sequence
  VL CDR3
QQGYSLRNIDNA

SEQ ID NO: 8 - protein, 166 aa, Artificial Sequence
  VH
METGLRWLLLVAVLKGVQCQSLEESGGRLVTPGTPLTLTCTASGFSLSNYYVTWVRQAPGKGLEWIGIIYGSDETAYATWAIGRFTISKTSTTVDLKMTSLTAADTATYFCARDDSSDWDAKFNLWGQGTLVTVSSASTKGPSVFPLAPSSKSTSGGTAALGCLVK

SEQ ID NO: 9 - protein, 109 aa, Artificial Sequence
  Another VH
EVQLVESGGGLVQPGGSLRLSCAASGFSLSNYYVTWVRQAPGKGLEWVGIIYGSDETAYATWAIGRFTISRDNSKNTLYLQMNSLRAEDTAVYYCARDDSSDWDAKFNL

SEQ ID NO: 10 - protein, 109 aa, Artificial Sequence
  Another VH
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNL

SEQ ID NO: 11 - protein, 163 aa, Artificial Sequence
  VL
MDTRAPTQLLGLLLLWLPGARCAYDMTQTPASVSAAVGGTVTIKCQASQSINNELSWYQQKPGQRPKLLIYRASTLASGVSSRFKGSGSGTEFTLTISDLECADAATYYCQQGYSLRNIDNAFGGGTEVVVKRTVAAPSVFIFPPSDEQLKSGTASVVCLLNN

SEQ ID NO: 12 - protein, 99 aa, Artificial Sequence
  Another VL
IQMTQSPSSLSASVGDRVTITCQASQSINNELSWYQQKPGKAPKLLIYRASTLASGVPSRFSGSGSGTDFTLTISSLQPDDFATYYCQQGYSLRNIDNA